(19) United States
(12) Patent Application Publication
(10) Pub. No.: US 2018/0155787 A1
(43) Pub. Date: Jun. 7, 2018

(54) NON-ALCOHOLIC FATTY LIVER DISEASE BIOMARKERS

(71) Applicants: REGULUS THERAPEUTICS INC., San Diego, CA (US); THE REGENTS OF THE UNIVERSITY OF CALIFORNIA, Oakland, CA (US)

(72) Inventors: Martin BEAULIEU, San Diego, CA (US); Nelson B CHAU, San Diego, CA (US); Vivek KAIMAL, San Diego (CA); Rohit LOOMBA, (US)

(21) Appl. No.: 15/579,523

(22) PCT Filed: Jun. 3, 2016
(86) PCT No.: PCT/US2016/035736

§ 371 Date: Dec. 4, 2017

Related U.S. Application Data
(60) Provisional application No. 62/171,726, filed on Jun. 5, 2015.

(51) Int. Cl. C12Q 1/6883; C12Q 1/6869; C12Q 1/6837; G01N 33/68; C12Q 1/686

(52) U.S. Cl. CPC C12Q 1/6883; C12Q 1/6869; C12Q 1/6837; C12Q 2600/118; C12Q 1/686; C12Q 2600/178; G01N 2800/085; G01N 33/6893

(57) ABSTRACT

Methods, compositions, kits, and systems for characterizing the non-alcoholic fatty liver disease (NAFLD) state of a subject are provided. In some embodiments the methods, compositions, kits, and systems comprise at least one miRNA selected from the differentially expressed miRNAs listed in at least one of Tables 1-4, 10-14, and 28-29. In some embodiments the methods compositions, kits, and systems are for characterizing the nonalcoholic steatohepatitis (NASH) state of the subject, characterizing the occurrence of liver fibrosis in the subject, and/or characterizing the occurrence of hepatocellular ballooning in the subject.

The instant application contains a Sequence Listing which has been submitted electronically in ASCII format and is hereby incorporated by reference in its entirety. Said ASCII copy, created on Jun. 2, 2016, is named 1007_002_PCT SL.txt and is 34,463 bytes in size.

### INTRODUCTION

Non-alcoholic fatty liver disease (NAFLD) is the buildup of extra fat in liver cells that is not caused by alcohol. It is normal for the liver to contain some fat. However, if more than 5%-10% percent of the liver's weight is fat, then it is called a fatty liver (steatosis). Many people have a buildup of fat in the liver, and for most people it causes no symptoms. NAFLD tends to develop in people who are overweight or obese or have diabetes, high cholesterol or high triglycerides. The most severe form of NAFLD is Nonalcoholic steatohepatitis (NASH). NASH causes scarring of the liver (fibrosis), which may lead to cirrhosis. NASH is similar to the kind of liver disease that is caused by long-term, heavy drinking. But NASH occurs in people who don't abuse alcohol. It is difficult to predict what NAFLD patient will develop NASH and often, people with NASH don't know they have it.

Liver biopsy is the gold standard for diagnosing NASH. The presence of fibrosis, lobular inflammation, steatosis and hepatocellular ballooning are key criteria used from histopathology data. There are no non-invasive NASH tests available. Currently, the detection of hepatocellular ballooning and steatosis is only achieved by histopathology from biopsy samples. For these and other reasons there is a need for new methods, systems, kits, and other tools for diagnosis and prognosis of NAFLD disease states including NASH, fibrosis, hepatocellar ballooning. Certain embodiments of this invention meets these and other needs.

### SUMMARY

The inventors have made the surprising discoveries that miRNAs are differentially expressed in the serum of subjects depending on the non-alcoholic fatty liver disease (NAFLD) state of the subject. These and other observations have, in part, allowed the inventors to provide herein methods, compositions, kits, and systems for characterizing the NAFLD state of the subject, as well as other inventions disclosed herein.

In some embodiments methods of characterizing the non-alcoholic fatty liver disease (NAFLD) state of a subject are provided. In some embodiments a method comprises forming a biomarker panel having N microRNAs (miRNAs) selected from the differentially expressed miRNAs listed in at least one of Tables 1-4, 10-14, and 28-29, and detecting the level of each of the N miRNAs in the panel in a sample from the subject. In some embodiments N is from 1 to 20, from 1 to 5, from 6 to 10, from 11 to 15, or from 15 to 20.

In some embodiments further methods of characterizing the NAFLD state in a subject are provided. In some embodiments a method comprises detecting the level of at least one, at least two, at least three, at least four, at least five, at least six, at least seven, at least eight, at least nine, or at least ten or at least 15 miRNAs selected from the differentially increased and differentially decreased miRNAs listed in at least one of Tables 1-4, 10-14, and 28-29 in a sample from the subject. In some embodiments a level of at least one differentially increased miRNA that is higher than a control level of the respective miRNA and/or a level of at least one differentially decreased miRNA that is lower than a control level of the respective miRNA indicates the presence of NAFLD and/or the presence of a more advanced NAFLD state in the subject. In some embodiments a level of at least one differentially increased miRNA that is higher than a control level of the respective miRNA and/or a level of at least one differentially decreased miRNA that is lower than a control level of the respective miRNA is detected and the subject is diagnosed as having NAFLD and/or a a more advanced NAFLD state. In some embodiments the method further comprises administering at least one NAFLD therapy to the subject based on the diagnosis.

In some embodiments methods of characterizing the NAFLD state of the subject comprise characterizing the nonalcoholic steatohepatitis (NASH) state of the subject. In some embodiments of methods the level of at least one, at least two, at least three, at least four, at least five, at least six, at least seven, at least eight, at least nine, or at least ten miRNAs selected from the differentially increased and differentially decreased miRNAs listed in at least one of Tables 1-4 is detected in the sample from the subject. In some embodiments a level of at least one differentially increased miRNA that is higher than a control level of the respective miRNA and/or a level of at least one differentially decreased miRNA that is lower than a control level of the respective miRNA indicates the presence of NASH and/or the presence of a more advanced stage of NASH in the subject. In some embodiments the NASH is stage 1, stage 2, stage 3 or stage 4 NASH. In some embodiments a level of at least one differentially increased miRNA that is higher than a control level of the respective miRNA and/or a level of at least one differentially decreased miRNA that is lower than a control level of the respective miRNA is detected and the subject is diagnosed as having NASH and/or a more advanced stage of NASH. In some embodiments the subject is diagnosed as having stage 1, stage 2, stage 3 or stage 4 NASH. In some embodiments the method further comprises administering at least one NASH therapy to the subject based on the diagnosis.

In some embodiments methods of characterizing the NAFLD state of the subject comprise characterizing the occurrence of liver fibrosis in the subject. In some embodiments of methods the level of at least one, at least two, at least three, at least four, at least five, at least six, at least seven, at least eight, at least nine, or at least ten miRNAs selected from the differentially increased and differentially decreased miRNAs listed in at least one of Tables 10-14 is detected in the sample from the subject. In some embodiments a level of at least one differentially increased miRNA that is higher than a control level of the respective miRNA and/or a level of at least one differentially decreased miRNA that is lower than a control level of the respective miRNA indicates the presence of liver fibrosis and/or the presence of more advanced liver fibrosis in the subject. In some embodiments a level of at least one differentially increased miRNA that is higher than a control level of the respective miRNA and/or a level of at least one differentially decreased miRNA that is lower than a control level of the respective miRNA is detected and the subject is diagnosed as having liver fibrosis and/or a more advanced liver fibrosis. In some embodiments the method further comprises administering at least one liver fibrosis therapy to the subject based on the diagnosis.

In some embodiments methods of characterizing the NAFLD state of the subject comprise characterizing the occurrence of hepatocellular ballooning in the subject. In some embodiments of methods detecting the level of at least one, at least two, at least three, at least four, at least five, at least six, at least seven, at least eight, at least nine, or at least ten miRNAs selected from the differentially increased and differentially decreased miRNAs listed in at least one of Tables 28 and 29 is detected in the sample from the subject. In some embodiments a level of at least one differentially increased miRNA that is higher than a control level of the respective miRNA and/or a level of at least one differentially decreased miRNA that is lower than a control level of the respective miRNA indicates the presence of hepatocellular ballooning and/or the presence of more advanced hepatocellular ballooning in the subject. In some embodiments a level of at least one differentially increased miRNA that is higher than a control level of the respective miRNA and/or a level of at least one differentially decreased miRNA that is lower than a control level of the respective miRNA is detected and the subject is diagnosed as having hepatocellular ballooning and/or more advanced hepatocellular ballooning. In some embodiments the method further comprises administering at least one hepatocellular ballooning therapy to the subject based on the diagnosis.

In some embodiments methods of determining whether a subject has NASH are provided. In some embodiments the methods comprise providing a sample from a subject suspected of having NASH; forming a biomarker panel having N miRNAs selected from the differentially increased and differentially decreased miRNAs listed in at least one of Tables 1-4; and detecting the level of each of the N miRNAs in the panel in the sample from the subject. In some embodiments N is from 1 to 20, from 1 to 5, from 6 to 10, from 11 to 15, or from 15 to 20. In some embodiments the methods comprise providing a sample from a subject suspected of NASH and detecting the level of at least one, at least two, at least three, at least four, at least five, at least six, at least seven, at least eight, at least nine, or at least ten miRNAs selected from the differentially increased and differentially decreased miRNAs listed in at least one of Tables 1-4 in the sample from the subject; wherein a level of at least one differentially increased miRNA that is higher than a control level of the respective miRNA and/or a level of at least one differentially decreased miRNA that is lower than a control level of the respective miRNA indicates that the subject has NASH. In some embodiments a method comprises detecting the level of at least one pair of miRNAs selected from pairs 1-10 listed in Table 5 in the sample from the subject. In some embodiments the sample is from a subject diagnosed with mild, moderate, or severe NAFLD. In some embodiments the subject is not previously diagnosed with NASH. In some embodiments the NASH is stage 1, 2, 3, or 4 NASH. In some embodiments the subject is previously diagnosed with NAFLD. In some embodiments the subject has presented with at least one clinical symptom of NASH. In some embodiments the methods comprise providing a sample from a subject suspected of NASH and detecting the level of at least one, at least two, at least three, at least four, at least five, at least six, at least seven, at least eight, at least nine, or at least ten miRNAs selected from the differentially increased and differentially decreased miRNAs listed in at least one of Tables 1-4 in the sample from the subject; wherein a level of at least one differentially increased miRNA that is higher than a control level of the respective miRNA and/or a level of at least one differentially decreased miRNA that is lower than a control level of the respective miRNA is detected and the subject is diagnosed as having NASH. In some embodiments the method further comprises administering at least one NASH therapy to the subject based on the diagnosis.

In some embodiments methods of monitoring NASH therapy in a subject are provided. In some embodiments a method comprises providing a sample from a subject undergoing treatment for NASH; forming a biomarker panel having N miRNAs selected from the differentially increased and differentially decreased miRNAs listed in at least one of Tables 1-4; and detecting the level of each of the N miRNAs in the panel in the sample from the subject. In some embodiments N is from 1 to 20, from 1 to 5, from 6 to 10, from 11 to 15, or from 15 to 20. In some embodiments the methods comprise providing a sample from a subject undergoing treatment for NASH and detecting the level of at least one, at least two, at least three, at least four, at least five, at least six, at least seven, at least eight, at least nine, or at least ten miRNAs selected from the differentially increased and differentially decreased miRNAs listed in at least one of Tables 1-4 in the sample from the subject; wherein a level of at least one differentially increased miRNA that is higher than a control level of the respective miRNA and/or a level of at least one differentially decreased miRNA that is lower than a control level of the respective miRNA indicates that the NASH is increasing in severity; and wherein the absence of a level of at least one differentially increased miRNA that is higher than a control level of the respective miRNA and/or a level of at least one differentially decreased miRNA that is lower than a control level of the respective miRNA indicates that the NASH is not increasing in severity. In some embodiments the methods comprise detecting the level of at least one pair of miRNAs selected from pairs 1-10 listed in Table 5 in the sample from the subject. In some embodiments the NASH is stage 1, 2, 3, or 4 NASH.

In some embodiments methods of characterizing the risk that a subject with NAFLD will develop NASH are provided. In some embodiments methods comprise providing a sample from a subject with NAFLD and detecting the level of at least one, at least two, at least three, at least four, at least five, at least six, at least seven, at least eight, at least nine, or at least ten miRNAs selected from the differentially increased and differentially decreased miRNAs listed in at least one of Tables 1-4 in the sample from the subject; wherein a level of at least one differentially increased miRNA that is higher than a control level of the respective miRNA and/or a level of at least one differentially decreased miRNA that is lower than a control level of the respective miRNA indicates an increased risk that the subject will develop NASH; and/or wherein the absence of a level of at least one differentially increased miRNA that is higher than a control level of the respective miRNA and/or a level of at least one differentially decreased miRNA that is lower than a control level of the respective miRNA indicates a decreased risk that the subject will develop NASH. In some embodiments a method comprises detecting the level of at least one pair of miRNAs selected from pairs 1-10 listed in Table 5 in the sample from the subject. In some embodiments the sample is from a subject diagnosed with mild, moderate, or severe NAFLD.

In some embodiments methods of determining whether a subject has liver fibrosis are provided. In some embodiments methods comprise providing a sample from a subject suspected of liver fibrosis; forming a biomarker panel having N miRNAs selected from the differentially increased and differentially decreased miRNAs listed in at least one of Tables 10-14; and detecting the level of each of the N miRNAs in the panel in the sample from the subject. In some embodiments N is from 1 to 20, from 1 to 5, from 6 to 10, from 11 to 15, or from 15 to 20. In some embodiments methods comprise determining whether a subject has liver fibrosis, comprising providing a sample from a subject suspected of having liver fibrosis and detecting the level of at least one, at least two, at least three, at least four, at least five, at least six, at least seven, at least eight, at least nine, or at least ten miRNAs selected from the differentially increased and differentially decreased miRNAs listed in at least one of Tables 10-14; wherein a level of at least one differentially increased miRNA that is higher than a control level of the respective miRNA and/or a level of at least one differentially decreased miRNA that is lower than a control level of the respective miRNA indicates the presence of liver fibrosis. In some embodiments a level of at least one differentially increased miRNA that is higher than a control level of the respective miRNA and/or a level of at least one differentially decreased miRNA that is lower than a control level of the respective miRNA is detected and the subject is diagnosed as having liver fibrosis. In some embodiments the method further comprises administering at least one liver fibrosis therapy to the subject based on the diagnosis. In some embodiments a method comprises detecting the level of at least one miRNA selected from the differentially increased and differentially decreased miRNAs listed in at least one of Tables 15-17. In some embodiments the at least one miRNA is miR-224. In some embodiments a method comprises detecting the level of at least one miRNA selected from the differentially increased and differentially decreased miRNAs listed in Table 18. In some embodiments a method comprises detecting the level of miR-224 and/or miR-191. In some embodiments the liver fibrosis is stage 1, 2, 3, or 4 liver fibrosis. In some embodiments the sample is from a subject diagnosed with mild, moderate, or severe NAFLD. In some embodiments the sample is from a subject diagnosed with NASH. In some embodiments the NASH is stage 1, 2, 3, or 4 NASH.

In some embodiments methods of determining whether a subject has hepatocellular ballooning are provided. In some embodiments methods comprise providing a sample from a subject suspected of having hepatocellular ballooning; forming a biomarker panel having N miRNAs selected from the differentially increased and differentially decreased miRNAs listed in at least one of Tables 28 and 29; and detecting the level of each of the N miRNAs in the panel in the sample from the subject. In some embodiments N is from 1 to 20, from 1 to 5, from 6 to 10, from 11 to 15, or from 15 to 20. In some embodiments methods comprise determining whether a subject has hepatocellular ballooning, comprising providing a sample from a subject suspected of having hepatocellular ballooning and detecting the level of at least one, at least two, at least three, at least four, at least five, at least six, at least seven, at least eight, at least nine, or at least ten miRNAs selected from the differentially increased and differentially decreased miRNAs listed in at least one of Tables 28 and 29 in the sample from the subject; wherein a level of at least one differentially increased miRNA that is higher than a control level of the respective miRNA and/or a level of at least one differentially decreased miRNA that is lower than a control level of the respective miRNA indicates the presence of hepatocellular ballooning. In some embodiments a level of at least one differentially increased miRNA that is higher than a control level of the respective miRNA and/or a level of at least one differentially decreased miRNA that is lower than a control level of the respective miRNA is detected and the subject is diagnosed as having hepatocellular ballooning. In some embodiments the method further comprises administering at least one hepatocellular ballooning therapy to the subject based on the diagnosis. In some embodiments a method comprises detecting the level of at least one pair of miRNAs selected from the pairs listed in Table 30 in the sample from the subject. In some embodiments a method comprises detecting the level of at least one pair of miRNAs selected from the pairs listed in Table 35 in the sample from the subject. In some embodiments the sample is from a subject diagnosed with mild, moderate, or severe NAFLD. In some embodiments the sample is from a subject diagnosed with NASH. In some embodiments the NASH is stage 1, 2, 3, or 4 NASH.

In some embodiments of the methods of this disclosure the method comprises detecting by a process comprising RT-PCR. In some embodiments the detecting comprises quantitative RT-PCR.

In some embodiments of the methods of this disclosure the sample is a bodily fluid. In some embodiments the sample is selected from blood, a blood component, urine, sputum, saliva, and mucus. In some embodiments the sample is serum.

In some embodiments of the methods of this disclosure the method comprises characterizing the NAFLD or NASH state of the subject for the purpose of determining a medical insurance premium or a life insurance premium. In some embodiments the method further comprises determining a medical insurance premium or a life insurance premium for the subject.

In some embodiments compositions are provided. In some embodiments a composition comprises RNAs of a sample from a subject or cDNAs reverse transcribed from the RNAs of a sample from a subject; and a set of polynucleotides for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 1-4, 10-14, and 28-29. In some embodiments the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 1-4. In some embodiments the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 10-14. In some embodiments the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 28 and 29. In some embodiments each polynucleotide in the composition independently comprises from 8 to 100, from 8 to 75, from 8 to 50, from 8 to 40, from 8 to 30, from 12 to 100, from 12 to 75, from 12 to 50, from 12 to 40, or from 12 to 30 nucleotides. In some embodiments the sample is a bodily fluid. In some embodiments the sample is selected from blood, a blood component, urine, sputum, saliva, and mucus. In some embodiments the sample is serum.

In some embodiments kits are provided. In some embodiments a kit comprises a set of polynucleotides for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 1-4, 10-14, and 28-29. In some embodiments the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 1-4. In some embodiments the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 10-14. In some embodiments the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 28 and 29. In some embodiments each polynucleotide in the kit independently comprises from 8 to 100, from 8 to 75, from 8 to 50, from 8 to 40, from 8 to 30, from 12 to 100, from 12 to 75, from 12 to 50, from 12 to 40, or from 12 to 30 nucleotides. In some embodiments the polynucleotides are packaged for use in a multiplex assay. In some embodiments the polynucleotides are packages for use in a non-multiplex assay.

In some embodiments systems are provided. In some embodiments a system comprises a set of polynucleotides for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 1-4, 10-14, and 28-29; and RNAs of a sample from a subject or cDNAs reverse transcribed from the RNAs of a sample from a subject. In some embodiments the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 1-4. In some embodiments the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 10-14. In some embodiments the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 28 and 29. In some embodiments each polynucleotide in the system independently comprises from 8 to 100, from 8 to 75, from 8 to 50, from 8 to 40, from 8 to 30, from 12 to 100, from 12 to 75, from 12 to 50, from 12 to 40, or from 12 to 30 nucleotides. In some embodiments the sample is a bodily fluid. In some embodiments the sample is selected from blood, a blood component, urine, sputum, saliva, and mucus. In some embodiments the sample is serum. In some embodiments the RNAs of a sample from a subject or cDNAs reverse transcribed from the RNAs of a sample from a subject are in a container, and wherein the set of polynucleotides is packaged separately from the container.

In some embodiments methods of detecting differential expression of miRNAs are provided. In some embodiments the method comprises providing a sample from a subject and detecting the level of at least one, at least two, at least three, at least four, at least five, at least six, at least seven, at least eight, at least nine, or at least ten or at least 15 miRNAs selected from the differentially increased and differentially decreased miRNAs listed in at least one of Tables 1-4, 10-14, and 28-29 in the sample from the subject. In some embodiments a level of at least one differentially increased miRNA that is higher than a control level of the respective miRNA and/or a level of at least one differentially decreased miRNA that is lower than a control level of the respective miRNA is detected. In some embodiments a level of at least one differentially increased miRNA that is higher than a control level of the respective miRNA and/or a level of at least one differentially decreased miRNA that is lower than a control level of the respective miRNA is not detected. In some embodments the subject is suspected of having NAFLD. In some embodments the subject is at risk of developing NAFLD. In some embodments the subject has NAFLD.

In some embodiments additional methods of detecting differential expression of miRNAs are provided. In some embodiments the method comprises providing a sample from a subject and detecting the level of at least one, at least two, at least three, at least four, at least five, at least six, at least seven, at least eight, at least nine, or at least ten miRNAs selected from the differentially increased and differentially decreased miRNAs listed in at least one of Tables 1-4 in the sample from the subject. In some embodiments a level of at least one differentially increased miRNA that is higher than a control level of the respective miRNA and/or a level of at least one differentially decreased miRNA that is lower than a control level of the respective miRNA is detected. In some embodiments a level of at least one differentially increased miRNA that is higher than a control level of the respective miRNA and/or a level of at least one differentially decreased miRNA that is lower than a control level of the respective miRNA is not detected. In some embodments the subject is suspected of having NASH. In some embodments the subject is at risk of developing NASH. In some embodments the subject has NASH. In some embodiments the NASH is stage 1, stage 2, stage 3 or stage 4 NASH. In some embodiments the method comprises detecting the level of at least one pair of miRNAs selected from pairs 1-10 listed in Table 5 in the sample from the subject.

In some embodiments additional methods of detecting differential expression of miRNAs are provided. In some embodiments the method comprises providing a sample from a subject and detecting the level of at least one, at least two, at least three, at least four, at least five, at least six, at least seven, at least eight, at least nine, or at least ten miRNAs selected from the differentially increased and differentially decreased miRNAs listed in at least one of Tables 10-14 is detected in the sample from the subject. In some embodiments a level of at least one differentially increased miRNA that is higher than a control level of the respective miRNA and/or a level of at least one differentially decreased miRNA that is lower than a control level of the respective miRNA is detected. In some embodiments a level of at least one differentially increased miRNA that is higher than a control level of the respective miRNA and/or a level of at least one differentially decreased miRNA that is lower than a control level of the respective miRNA is not detected. In some embodments the subject is suspected of having liver fibrosis. In some embodments the subject is at risk of developing liver fibrosis. In some embodments the subject has liver fibrosis. In some embodiments the method comprises detecting the level of at least one miRNA selected from the differentially increased and differentially decreased miRNAs listed in at least one of Tables 15-17. In some embodiments the at least one miRNA is miR-224. In some embodiments the method comprises detecting the level of at least one miRNA selected from the differentially increased and differentially decreased miRNAs listed in Table 18. In some embodiments the method comprises detecting the level of miR-224 and/or miR-191.

In some embodiments additional methods of detecting differential expression of miRNAs are provided. In some embodiments the method comprises providing a sample from a subject and detecting the level of at least one, at least two, at least three, at least four, at least five, at least six, at least seven, at least eight, at least nine, or at least ten miRNAs selected from the differentially increased and differentially decreased miRNAs listed in at least one of Tables 28 and 29 in the sample from the subject. In some embodiments a level of at least one differentially increased miRNA that is higher than a control level of the respective miRNA and/or a level of at least one differentially decreased miRNA that is lower than a control level of the respective miRNA is detected. In some embodiments a level of at least one differentially increased miRNA that is higher than a control level of the respective miRNA and/or a level of at least one differentially decreased miRNA that is lower than a control level of the respective miRNA is not detected. In some embodments the subject is suspected of having hepatocellular ballooning. In some embodments the subject is at risk of developing hepatocellular ballooning. In some embodments the subject has hepatocellular ballooning. In some embodiments the method comprises detecting the level of at least one pair of miRNAs selected from the pairs listed in Table 30 in the sample from the subject. In some embodiments the method comprises detecting the level of at least one pair of miRNAs selected from the pairs listed in Table 35 in the sample from the subject.

### BRIEF DESCRIPTION OF THE DRAWINGS

FIG. 1 shows a Venn diagram depicting the number of miRNAs modulated between different stages of fibrosis.

### TABLES

Tables 1-39 are presented together at the end of the specification. Those tables are referenced in the text of the application and form a part of the application.

### DESCRIPTION

While the invention will be described in conjunction with certain representative embodiments, it will be understood that the invention is defined by the claims, and is not limited to those embodiments.

One skilled in the art will recognize that many methods and materials similar or equivalent to those described herein may be used in the practice of the present invention. The present invention is in no way limited to the methods and materials literaly described.

Unless defined otherwise, technical and scientific terms used herein have the meaning commonly understood by one of ordinary skill in the art to which this invention belongs. Although any methods, devices, and materials similar or equivalent to those described herein can be used in the practice of the invention, certain methods, devices, and materials are described herein.

All publications, published patent documents, and patent applications cited herein are hereby incorporated by reference to the same extent as though each individual publication, published patent document, or patent application was specifically and individually indicated as being incorporated by reference.

As used in this application, including the appended claims, the singular forms “a,” “an,” and “the” include the plural, unless the context clearly dictates otherwise, and may be used interchangeably with “at least one” and “one or more.” Thus, reference to “a miRNA” includes mixtures of miRNAs, and the like.

As used herein, the terms “comprises,” “comprising,” “includes,” “including,” “contains,” “containing,” and any variations thereof, are intended to cover a non-exclusive inclusion, such that a process, method, product-by-process, or composition of matter that comprises, includes, or contains an element or list of elements may include other elements not expressly listed.

The present application includes biomarkers, methods, devices, reagents, systems, and kits for determining whether a subject has NAFLD. The present application also includes biomarkers, methods, devices, reagents, systems, and kits for determining whether a subject has NASH. In some embodiments, biomarkers, methods, devices, reagents, systems, and kits are provided for determining whether a subject with NAFLD has NASH. The present application also includes biomarkers, methods, devices, reagents, systems, and kits for determining whether a subject has liver fibrosis. The present application also includes biomarkers, methods, devices, reagents, systems, and kits for determining whether a subject has hepatocellular ballooning.

As used herein, “nonalcoholic fatty liver disease” or “NAFLD” refers to a condition in which fat is deposited in the liver (hepatic steatosis), with or without inflammation and fibrosis, in the absence of excessive alcohol use.

As used herein, “nonalcoholic steatohepatitis” or “NASH” refers to NAFLD in which there is inflammation and/or fibrosis in the liver. NASH may be divided into four stages. Exemplary methods of determining the stage of NASH are described, for example, in Kleiner et al, 2005, Hepatology, 41(6): 1313-1321, and Brunt et al, 2007, Modern Pathol, 20: S40-S48.

As used herein, “liver fibrosis” refers to formation of excess fibrous connective tissue in the liver.

As used herein, “hepatocellular ballooning” refers to the process of hepatocyte cell death.

“MicroRNA” means an endogenous non-coding RNA between 18 and 25 nucleobases in length, which is the product of cleavage of a pre-microRNA by the enzyme Dicer. Examples of mature microRNAs are found in the microRNA database known as miRBase (http://microrna.sanger.ac.uk/). In certain embodiments, microRNA is abbreviated as “microRNA” or “miRNA” or “miR. Several exemplary miRNAs are provided herein identified by their common name and their nucleobase sequence.

“Pre-microRNA” or “pre-miRNA” or “pre-miR” means a non-coding RNA having a hairpin structure, which is the product of cleavage of a pri-miR by the double-stranded RNA-specific ribonuclease known as Drosha.

“Stem-loop sequence” means an RNA having a hairpin structure and containing a mature microRNA sequence. Pre-microRNA sequences and stem-loop sequences may overlap. Examples of stem-loop sequences are found in the microRNA database known as miRBase. (http://microrna.sanger.ac.uld).

“Pri-microRNA” or “pri-miRNA” or “pri-miR” means a non-coding RNA having a hairpin structure that is a substrate for the double-stranded RNA-specific ribonuclease Drosha.

“microRNA precursor” means a transcript that originates from a genomic DNA and that comprises a non-coding, structured RNA comprising one or more microRNA sequences. For example, in certain embodiments a microRNA precursor is a pre-microRNA. In certain embodiments, a microRNA precursor is a pri-microRNA.

Some of the methods of this disclosure comprise detecting the level of at least one miRNA in a sample. In some embodiments the sample is a bodily fluid. In some embodiments the bodily fluid is selected from blood, a blood component, urine, sputum, saliva, and mucus. In some embodiments the samle is serum. Detecting the level in a sample encompasses methods of detecting the level directly in a raw sample obtained from a subject and also methods of detecting the level following processing of the sample. In some embodiments the raw sample is processed by a process comprising enriching the nucleic acid in the sample relative to other components and/or enriching small RNAs in the sample relative to other components.

In embodiments, detecting the level of a miRNA in a sample may be by a method comprising direct detection of miRNA molecules in the sample. In embodiments, detecting the level of a miRNA in a sample may be by a method comprising reverse transcribing part or all of the miRNA molecule and then detecting a cDNA molecule and/or detecting a molecule comprising a portion corresponding to original miRNA sequence and a portion corresponding to cDNA.

Any suitable method known in the art may be used to detect the level of the at least one miRNA. One class of such assays involves the use of a microarray that includes one or more aptamers immobilized on a solid support. The aptamers are each capable of binding to a target molecule in a highly specific manner and with very high affinity. See, e.g., U.S. Pat. No. 5,475,096 entitled “Nucleic Acid Ligands”; see also, e.g., U.S. Pat. No. 6,242,246, U.S. Pat. No. 6,458,543, and U.S. Pat. No. 6,503,715, each of which is entitled “Nucleic Acid Ligand Diagnostic Biochip”. Once the microarray is contacted with a sample, the aptamers bind to their respective target molecules present in the sample and thereby enable a determination of a miRNA level corresponding to a miRNA in the sample.

As used herein, an “aptamer” refers to a nucleic acid that has a specific binding affinity for a target molecule, such as a miRNA or a cDNA encoded by a miRNA. It is recognized that affinity interactions are a matter of degree; however, in this context, the “specific binding affinity” of an aptamer for its target means that the aptamer binds to its target generally with a much higher degree of affinity than it binds to other components in a test sample. An “aptamer” is a set of copies of one type or species of nucleic acid molecule that has a particular nucleotide sequence. An aptamer can include any suitable number of nucleotides, including any number of chemically modified nucleotides. “Aptamers” refers to more than one such set of molecules. Different aptamers can have either the same or different numbers of nucleotides. Aptamers can be DNA or RNA or chemically modified nucleic acids and can be single stranded, double stranded, or contain double stranded regions, and can include higher ordered structures. As further described below, an aptamer may include a tag. If an aptamer includes a tag, all copies of the aptamer need not have the same tag. Moreover, if different aptamers each include a tag, these different aptamers can have either the same tag or a different tag.

As used herein, a “differentially regulated” miRNA is an miRNA that is increased or decreased in abundance in a sample from a subject having a disease or condition of interest in comparison to a control level of the miRNA that occurs in a similar sample from a subject not having the disease or condition of interest. The subject not having the disease or condition of interest may be a subject that does not have any related disease or condition (e.g., a normal control subject) or the subject may have a different related disease or condition (e.g., a subject having NAFLD but not having NASH).

As used herein a “differentially increased” miRNA is an miRNA that is increased in abundance in a sample from a subject having a disease or condition of interest in comparison to the level of the miRNA that occurs in a control sample from a subject not having the disease or condition of interest.

As used herein a “differentially decreased” miRNA is an miRNA that is decreased in abundance in a sample from a subject having a disease or condition of interest in comparison to the level of the miRNA that occurs in a control sample from a subject not having the disease or condition of interest.

As used herein a “control level” of an miRNA is the level that is present in similar samples from a reference population. A “control level” of a miRNA need not be determined each time the present methods are carried out, and may be a previously determined level that is used as a reference or threshold to determine whether the level in a particular sample is higher or lower than a normal level. In some embodiments, a control level in a method described herein is the level that has been observed in one or more subjects without NAFLD. In some embodiments, a control level in a method described herein is the level that has been observed in one or more subjects with NAFLD, but not NASH. In some embodiments, a control level in a method described herein is the average or mean level, optionally plus or minus a statistical variation, that has been observed in a plurality of normal subjects, or subjects with NAFLD but not NASH.

As used herein, “individual” and “subject” are used interchangeably to refer to a test subject or patient. In various embodiments, the individual is a mammal. A mammalian individual can be a human or non-human. In various embodiments, the individual is a human. A healthy or normal individual is an individual in which the disease or condition of interest (such as NASH) is not detectable by conventional diagnostic methods.

“Diagnose,” “diagnosing,” “diagnosis,” and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose,” “diagnosing,” “diagnosis,” etc., encompass, with respect to a particular disease or condition, the initial detection of the disease; the characterization or classification of the disease; the detection of the progression, remission, or recurrence of the disease; and/or the detection of disease response after the administration of a treatment or therapy to the individual. The diagnosis of NAFLD includes distinguishing individuals who have NAFLD from individuals who do not. The diagnosis of NASH includes distinguishing individuals who have NASH from individuals who have NAFLD, but not NASH, and from individuals with no liver disease. The diagnosis of liver fibrosis includes distinguishing individuals who have liver fibrosis from individuals who have NAFLD but do not have liver fibrosis. The diagnosis of hepatocellular ballooning includes distinguishing individuals who have hepatocellular ballooning from individuals who have NAFLD but do not have hepatocellular ballooning.

“Prognose,” “prognosing,” “prognosis,” and variations thereof refer to the prediction of a future course of a disease or condition in an individual who has the disease or condition (e.g., predicting disease progression), and prediction of whether an individual who does not have the diease or condition will develop the disease or condition. Such terms also encompass the evaluation of disease response after the administration of a treatment or therapy to the individual.

“Characterize,” “characterizing,” “characterization,” and variations thereof encompass both “diagnose” and “prognose” and also encompass determinations or predictions about the future course of a disease or condition in an individual who does not have the disease as well as determinations or predictions regarding the likelihood that a disease or condition will recur in an individual who apparently has been cured of the disease. The term “characterize” also encompasses assessing an individual's response to a therapy, such as, for example, predicting whether an individual is likely to respond favorably to a therapeutic agent or is unlikely to respond to a therapeutic agent (or will experience toxic or other undesirable side effects, for example), selecting a therapeutic agent for administration to an individual, or monitoring or determining an individual's response to a therapy that has been administered to the individual. Thus, “characterizing” NAFLD can include, for example, any of the following: prognosing the future course of NAFLD in an individual; predicting whether NAFLD will progress to NASH; predicting whether a particular stage of NASH will progress to a higher stage of NASH; predicting whether an individial with NAFLD will develop liver fibrosis; predicting whether a particular state of liver fibrosis will progress to the next state of liver fibrosis; predicting whether an individial with NAFLD will develop hepatocellular ballooning, etc.

As used herein, “detecting” or “determining” with respect to a miRNA level includes the use of both the instrument used to observe and record a signal corresponding to a miRNA level and the material/s required to generate that signal. In various embodiments, the level is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

As used herein, a “subject with NAFLD” refers to a subject that has been diagnosed with NAFLD. In some embodiments, NAFLD is suspected during a routine checkup, monitoring of metabolic syndrome and obesity, or monitoring for possible side effects of drugs (e.g., cholesterol lowering agents or steroids). In some instance, liver enzymes such AST and ALT are high. In some embodiments, a subject is diagnosed following abdominal or thoracic imaging, liver ultrasound, or magnetic resonance imaging. In some embodiments, other conditions such as excess alcohol consumption, hepatitis C, and Wilson's disease have been ruled out prior to an NAFLD diagnosis. In some embodiments, a subject has been diagnosed following a liver biopsy.

As used herein, a “subject with NASH” refers to a subject that has been diagnosed with NASH. In some embodiments, NASH is diagnosed by a method described above for NAFLD in general. In some embodiments, advanced fibrosis is diagnosed in a patient with NAFLD, for example, according to Gambino R, et. al. Annals of Medicine 2011; 43(8):617-49.

As used herein, a “subject at risk of developing NAFLD”” refers to a subject with one or more NAFLD comorbidities, such as obesity, abdominal obesity, metabolic syndrome, cardiovascular disease, and diabetes.

As used herein, a “subject at risk of developing NASH” refers to a subject with steatosis who continues to have one or more NAFLD comorbidities, such as obesity, abdominal obesity, metabolic syndrome, cardiovascular disease, and diabetes.

In some embodiments, the number and identity of miRNAs in a panel are selected based on the sensitivity and specificity for the particular combination of miRNA biomarker values. The terms “sensitivity” and “specificity” are used herein with respect to the ability to correctly classify an individual, based on one or more miRNA levels detected in a biological sample, as having the disease or not having the disease. In some embodiments, the terms “sensitivity” and “specificity” may be used herein with respect to the ability to correctly classify an individual, based on one or more miRNA levels detected in a biological sample, as having or not having the disease or condition. In such embodiments, “sensitivity” indicates the performance of the miRNAs with respect to correctly classifying individuals having the disease or condition. “Specificity” indicates the performance of the miRNAs with respect to correctly classifying individuals who do not have the disease or condition. For example, 85% specificity and 90% sensitivity for a panel of miRNAs used to test a set of control samples (such as samples from healthy individuals or subjects known not to have NASH) and test samples (such as samples from individuals with NASH) indicates that 85% of the control samples were correctly classified as control samples by the panel, and 90% of the test samples were correctly classified as test samples by the panel.

Any combination of the miRNAs described herein can be detected using a suitable kit, such as a kit for use in performing the methods disclosed herein. Furthermore, any kit can contain one or more detectable labels as described herein, such as a fluorescent moiety, etc. In some embodiments, a kit includes (a) one or more reagents for detecting one or more miRNAs in a biological sample, and optionally (b) one or more software or computer program products for predicting whether the individual from whom the biological sample was obtained has NAFLD, NASH (such as stage 1, 2, 3, or 4 NASH, or stage 2, 3, or 4 NASH, or stage 3 or 4 NASH), liver fibrosis (such as stage 1, 2, 3, or 4 fibrosis, or stage 3 or 4 fibrosis). Alternatively, rather than one or more computer program products, one or more instructions for manually performing the above steps by a human can be provided.

In some embodiments, a kit comprises at least one polynucleotide that binds specifically to at least one miRNA sequence disclosed herein. In some embodiments the kit futher comprises a signal generating material. The kit can also include instructions for using the devices and reagents, handling the sample, and analyzing the data. Further the kit may be used with a computer system or software to analyze and report the result of the analysis of the biological sample.

The kits can also contain one or more reagents (e.g., solubilization buffers, detergents, washes, or buffers) for processing a biological sample. Any of the kits described herein can also include, e.g., buffers, positive control samples, negative control samples, software and information such as protocols, guidance and reference data.

In some embodiments, kits are provided for the analysis of NAFLD and/or NASH and/or liver fibrosis and/or hepatocellular ballooning, wherein the kits comprise PCR primers for amplification of one or more miRNAs described herein. In some embodiments, a kit may further include instructions for use and correlation of the miRNAs with NAFLD and/or NASH and/or liver fibrosis and/or hepatocellular ballooning diagnosis and/or prognosis. In some embodiments, a kit may include a DNA array containing the complement of one or more of the miRNAs described herein, reagents, and/or enzymes for amplifying or isolating sample DNA. The kits may include reagents for real-time PCR such as quantitative real-time PCT.

### EXAMPLES

The following examples are provided for illustrative purposes only and are not intended to limit the scope of the invention as defined by the appended claims or as otherwise described herein.

### Example 1: Isolating Small RNAs from Serum

The following reagents and equipment were used to isolate small RNAs, including miRNAs, from human serum samples.

| Reagent | Vendor | P/N |
|---|---|---|
| Qiazol | Qiagen | 79306 |
| Chloroform (mol.bio grade) | MP Biomedicals | 194002 |
| Ath-159a (spike-in control) | IDT | 56017042 |
| 50 ml conical tubes | VWR | 21008-178 |
| 2 ml Non-stick micro-centrifuge | Ambion/Life Tech | AM12475 |
| tubes | | |
| Table top micro-centrifuge | Eppendorf | 5417R |
| refrigerated | | |
| Multi-tube vortexer | Fisher-Scientific | 02-215-450 |
| Table top centrifuge (Sorval | Thermo-Scientific | 75004521 |
| Legend XT) | | |
| Speed-vac (Savant) | Thermo-Scientific | DNA 120-115 |
| non-skirted 96-well pcr plates | Thermo-Scientific | AB-0600 |
| 48-well deep well plates | VWR | 12000-728 |
| Eppendorf Repeater Plus | VWR | 21516-002 |
| miRNeasy 96 Kit | Qiagen | 217061 |
| Reservoirs sterile Individually | VWR | 89094-678 |
| wrapped | | |
| 12-well multi-channel 1.2 ml | Rainin | L12-1200XLS |
| pipette LTS | | |
| 12-well multi-channel 200 ul | Rainin | L12-200XLS |
| pipette LTS | | |
| 12-well multi-channel 20 ul pipette | Rainin | L12-20XLS |
| LTS | | |
| Eppendorf Repeater Plus | VWR | 21516-002 |
| Reservoirs sterile Individually | VWR | 89094-678 |
| wrapped | | |
| 1 ml pipette LTS | Rainin | L-1000XLS |
| 200 ul pipette LTS | Rainin | L-200XLS |
| 20 ul pipette LTS | Rainin | L-20XLS |

140 uL of serum was extracted using the miRNeasy 96 Kit (Qiagen, cat. no. 217061) and following manufacturer's instructions:

### Example 2: MicroRNA Profiling Using Open Array Platform

The following reagents and equipment were used to profile miRNAs using an open array platform:

| Reagent | Vendor | P/N |
|---|---|---|
| TaqMan ® OpenArray ® Human miRNA Panel | Life Tech | 4470187 |
| OpenArray ® 384-well Sample Plates | Life Tech | 4406947 |
| OpenArray ® AccuFill ™ System Tips | Life Tech | 4457246 |
| OpenArray ® AccuFill ™ System Tips, 10 pack | Life Tech | 4458107 |
| TaqMan ® OpenArray ® Real-Time Master Mix, 5 mL | Life Tech | 4462164 |
| TaqMan ® OpenArray ® Real-Time PCR Accessories Kit | Life Tech | 4453993 |
| Megaplex ™ Primer Pools, Human Pool A v2.1 | Life Tech | 439996 |
| Megaplex ™ Primer Pools, Human Pool B v3.0 | Life Tech | 4444281 |
| TaqMan ® PreAmp Master Mix | Life Tech | 4391128 |
| TaqMan ® MicroRNA Reverse Transcription Kit, 1000 rxns | Life Tech | 4366597 |
| TaqMan PreAmp Master Mix | Life Tech | 4391128 |
| Taqman MegaPlex PreAmp Primers, Human Pool 1 v2.1 | Life Tech | 4399233 |
| Taqman MegaPlex PreAmp Primers, Human Pool 1 3.0 | Life Tech | 4444303 |
| StepOnePlus PCR machine or equivalent | Life Tech | 4376600 |

The following procedures were used:

Reverse Transcription (RT):

Four uL of RNA from example 1 was submitted to reverse transcription using Megaplex™ Primer Pools, Human Pool A v2.1 (439996) and a second 4 uL RNA was submitted to reverse transcription using Megaplex™ Primer Pools, Human Pool B v3.0 (Life Tech 4444281). The manufacturer's instructions were followed for 10 uL total reaction volume. The thermal cycling parameters were as follows.

### Reverse Transcription Thermal Cycler Protocol

| Stage | Temp | Time |
|---|---|---|
| Cycle (40 Cycles) | 16 C. | 2 min |
| | 42 C. | 1 min |
| | 50 C. | 1 sec |
| HOLD | 85 C. | 5 min |
| HOLD | 4 C. | ∞ |

Pre-Amplification of RT Samples:

Pre-amplification of reverse transcription products was achieved using their respective pre-amplification reagents for panel A and panel B, following the manufacturer's instructions to achieve a 40 uL reaction. The following thermal cycling parameters were used.

### Pre-Amplification Thermal Cycler Protocol

| Stage | Temp | Time |
|---|---|---|
| HOLD | 95 | 10 min |
| HOLD | 55 | 2 min |
| HOLD | 72 | 2 min |
| 16 cycles | 95 | 15 sec |
| | 60 | 4 min |
| HOLD | 99 | 10 min |
| HOLD | 4 | ∞ |

Real-Time qPCR Analysis.

Three ul of Pre-Amp cDNA (RT reaction product above) were diluted into 117u1 of RNAse, DNAse-free H₂O. Thirty uL of the diluted cDNA were transferred into a 96 well plate containing 30 uL of Open Array Master Mix prepared as per Manufacturer's instructions (Life Technologies). The mixture was loaded onto an TaqMan® OpenArray® Human MicroRNA Panel (4470187, Life Tech) using an QuantStudio™ 12K Flex Accufill System (4471021, Life Tech). The plate was loaded into an Applied Biosystems QuantStudio™ 12K Flex Real-Time PCR System (4471090, Life Tech) and real-time amplification was initiated using the following thermal cycling parameters.

### Real-Time uPCR Thermal Cycler Protocol

| Stage | Temp | Time |
|---|---|---|
| HOLD | 50 | 2 min |
| HOLD | 95 | 10 min |
| 40 cycles | 95 | 15 sec |
| | 60 | 1 min |

### Example 3: Serum Samples from NAFLD Patients

Frozen serum samples from 156 NAFLD patients were obtained and initially profiled using the OpenArray® Real-Time PCR System (ThermoFisher) using the procedures described in Examples 1 and 2. The raw PCR data were filtered, Ct values less than 10 were ignored, and Ct values above 28 were either ignored or set to 28. The subsequent analyses applied both sets of values. The filtered data were normalized by geometric mean of detected miRNAs.

These filtered, normalized values were used in exploratory analyses. Principal component analysis (PCA) was applied to discover technical and biological biases in miRNA expression data. PCA outliers such as samples with potentially degraded RNA were excluded. A total of 153 NAFLD samples passed these procedures; these were used in discovery of multi-miRNA classifiers that separates NAFL serum samples from NASH serum samples. As well, fibrosis grades, steatosis and hepatocellular ballooning were used to discover classifiers that separated the respective grades.

PCA analysis revealed no strong correlation between the profiles and categorical clinical parameters like gender, race, ethnicity, smoking, Diabetic Mellitus (DM), steatosis, fibrosis, lobular inflammation, portal inflammation, hepatocellular ballooning, NAFLD Activity Score (NAS), portal triads and clinical NAFL classification (data now shown). Only the third principal component, which accounts for <10% of variance in the data, was statistically significantly associated with categorical variables like hepatocellular ballooning, NAFL classification, NAS, steatosis and fibrosis (data not shown).

### Example 4: Identification of MicroRNAs Differentially Expressed in NASH

The 153 samples were classified into each of the following categories: NASH 3 (114), Borderline/Suspicious 2 (17), NAFLD 1 (18), and non-NAFLD 0 (2), using the classification criteria and procedures described in Kleiner et al, 2005, Hepatology, 41(6): 1313-1321. Two samples had no NAFL/NASH classification available.

Table 1 presents mean NASH vs. NAFLD differential expression data for 33 miRNAs that are differentially expressed in serum samples obtained from patients NASH patients and serum samples obtained from NAFLD patients without NASH. 23 of the miRNAs are decreased in serum samples obtained from patients having a NASH diagnosis relative to their expression level in serum samples obtained from NAFLD patients diagnosed as free of NASH. 10 of the miRNAs are increased in serum samples obtained from patients having a NASH diagnosis relative to their expression level in serum samples obtained from NAFLD patients diagnosed as free of NASH.

Table 2 presents mean NASH 3 vs. NAFLD 1 differential expression data for 24 miRNAs that are differentially expressed in serum samples obtained from patients diagnosed with NASH 3 compared to serum samples obtained from patients diagnosed with NAFLD 1. 17 of the miRNAs are decreased in serum samples obtained from patients having a diagnosis of NASH 3 relative to their expression level in serum samples obtained from patients having a diagnosis of NAFLD 1. 7 of the miRNAs are increased in serum samples obtained from patients having a diagnosis of NASH 3 relative to their expression level in serum samples obtained from patients having a diagnosis of NAFLD 1.

Table 3 presents mean NASH 3 vs. borderline 2 differential expression data for 17 miRNAs that are differentially expressed in serum samples obtained from patients diagnosed with NASH 3 compared to serum samples obtained from patients diagnosed with borderline 2. 9 of the miRNAs are decreased in serum samples obtained from patients having a diagnosis of NASH 3 relative to their expression level in serum samples obtained from patients having a diagnosis of borderline 2. 8 of the miRNAs are increased in serum samples obtained from patients having a diagnosis of NASH 3 relative to their expression level in serum samples obtained from patients having a diagnosis of borderline 2.

Table 4 presents mean borderline 2 vs. NAFLD 1 differential expression data for 10 miRNAs that are differentially expressed in serum samples obtained from patients diagnosed with borderline 2 compared to serum samples obtained from patients diagnosed with NAFLD 1. 5 of the miRNAs are decreased in serum samples obtained from patients having a diagnosis of borderline 2 relative to their expression level in serum samples obtained from patients having a diagnosis of NAFLD 1. 5 of the miRNAs are increased in serum samples obtained from patients having a diagnosis of borderline 2 relative to their expression level in serum samples obtained from patients having a diagnosis of NAFLD 1.

The data presented in Tables 1-4 identifies sets of miRNAs that are differentially expressed in serum samples obtained from patients having different NAFLD and NASH disease states. The identified miRNAs may be used individually or in combination as biomarkers to identify the disease state of a patient based on determining the miRNA expression profile of the selected miRNAs in a serum sample of a patient.

### Example 5: MicroRNA Expression Classifier for NASH Vs. NAFLD

Serum microRNA profiles were classified into NASH or NAFL using the following binary classifiers: Compound Covariate Predictor, Diagonal Linear Discriminant Analysis, and/or Support Vector Machines. The number of microRNAs was set to 20 (10 pairs). These 10 pairs of microRNAs were identified using the greedy-pairs approach (Bo et al. 2002). The greedy-pairs method starts by ranking all microRNAs based on individual t-scores. The best-ranked microRNA is selected, and the procedure then searches for the microRNA that together with the best-ranked microRNA provides the best discrimination and maximizes the pair t-score. The pair is then removed from the set of microRNAs, and the process is repeated on the remaining set of microRNAs until the desired number of pairs of microRNAs is reached. The desired number of pairs is specified a priori. Various numbers of pairs were specified and the one with the best AUC was picked. The notion behind the greedy-pairs method is that methods that would consider each microRNA separately may miss sets of microRNAs that together separate classes well, but not so well individually (Bo et al. 2002). This procedure identified the ten pair classifier identified in Table 5. The gene weights for the twenty miRNAs for each of the binary classifiers are provided in Table 6.

Prediction Rule from the 3 Classification Methods:

The prediction rule is defined by the inner sum of the weights (wᵢ) and expression (xᵢ) of significant genes. The expression is the log ratios for dual-channel data and log intensities for single-channel data.

A sample is classified to the class NAFL if the sum is greater than the threshold; that is,

Σᵢwᵢxᵢ>threshold

The threshold for the Compound Covariate predictor is −237.511. The threshold for the Diagonal Linear Discriminant predictor is −71.996. The threshold for the Support Vector Machine predictor is 26.091.

Cross-validation was used to test the performance of the classifiers, as follows.

Let, for some class A,

n11=number of class A samples predicted as A,
n12=number of class A samples predicted as non-A,
n21=number of non-A samples predicted as A,
n22=number of non-A samples predicted as non-A.

Then the following parameters can characterize performance of classifiers:

Sensitivity=n11/(n11+n12),

Specificity=n22/(n21+n22),

Positive Predictive Value(PPV)=n11/(n11+n21),

Negative Predictive Value(NPV)=n22/(n12+n22).

Sensitivity is the probability for a class A sample to be correctly predicted as class A. Specificity is the probability for a non class A sample to be correctly predicted as non-A. PPV is the probability that a sample predicted as class A actually belongs to class A. NPV is the probability that a sample predicted as non class A actually does not belong to class A.

The performance of the Compound Covariate Predictor Classifier is presented in Table 7. The performance of the Diagonal Linear Discriminant Analysis Classifier is presented in Table 8. The performance of the Support Vector Machine Classifier is presented in Table 9.

The receiver operator characteristic (ROC) of the classifier were represented graphically. The area under the curve (AUC) obtained averaged 0.68 using 3 classification methods: AUC of 0.676 obtained by Compound Covariate Predictor (CCP), AUC 0.708 obtained by Diagonal Linear Discriminant Predictor (DLDP) and AUC of 0.669 obtained by Bayesian Compound Covariate Predictor (BCCP).

### Example 6: Identification of MicroRNAs Differentially Expressed in Liver Fibrosis

The 153 NAFLD samples described in Example 3 were classified into each of the following categories: 62 (as well as the 2 non-NAFLD samples) had no fibrosis (Stage 0). The 2 samples with unknown NAFL score also had no fibrosis (Stage 0). 51 samples had fibrosis Stage 1, 16 had fibrosis Stage 2, 12 had fibrosis Stage 3, and 10 had fibrosis Stage 4.

Table 10 presents mean fibrosis stage 3 & 4 vs. fibrosis free differential expression data for 28 miRNAs that are differentially expressed in serum samples obtained from patients diagnosed with stage 3 or stage 4 fibrosis and serum samples obtained from patients diagnosed as free of fibrosis. 15 of the miRNAs are decreased in serum samples obtained from patients having a stage 3 or stage 4 fibrosis diagnosis relative to their expression level in serum samples obtained from patients diagnosed as free of fibrosis. 13 of the miRNAs are increased in serum samples obtained from patients having a stage 3 or stage 4 fibrosis diagnosis relative to their expression level in serum samples obtained from patients diagnosed as free of fibrosis.

Table 11 presents mean fibrosis stage 2 vs. fibrosis free differential expression data for 30 miRNAs that are differentially expressed in serum samples obtained from patients diagnosed with stage 2 fibrosis and serum samples obtained from patients diagnosed as free of fibrosis. 15 of the miRNAs are decreased in serum samples obtained from patients having a stage 2 fibrosis diagnosis relative to their expression level in serum samples obtained from patients diagnosed as free of fibrosis. 15 of the miRNAs are increased in serum samples obtained from patients having a stage 2 fibrosis diagnosis relative to their expression level in serum samples obtained from patients diagnosed as free of fibrosis.

Table 12 presents mean fibrosis stage 1 vs. fibrosis free differential expression data for 16 miRNAs that are differentially expressed in serum samples obtained from patients diagnosed with stage 1 fibrosis and serum samples obtained from patients diagnosed as free of fibrosis. 10 of the miRNAs are decreased in serum samples obtained from patients having a stage 1 fibrosis diagnosis relative to their expression level in serum samples obtained from patients diagnosed as free of fibrosis. 6 of the miRNAs are increased in serum samples obtained from patients having a stage 1 fibrosis diagnosis relative to their expression level in serum samples obtained from patients diagnosed as free of fibrosis.

Table 13 presents mean fibrosis stage 1 & 2 vs. fibrosis free differential expression data for 25 miRNAs that are differentially expressed in serum samples obtained from patients diagnosed with stage 1 or stage 2 fibrosis and serum samples obtained from patients diagnosed as free of fibrosis. 14 of the miRNAs are decreased in serum samples obtained from patients having a stage 1 or stage 2 fibrosis diagnosis relative to their expression level in serum samples obtained from patients diagnosed as free of fibrosis. 11 of the miRNAs are increased in serum samples obtained from patients having a stage 1 or stage 2 fibrosis diagnosis relative to their expression level in serum samples obtained from patients diagnosed as free of fibrosis.

Table 14 presents mean fibrosis stage 1/2 vs. mean fibrosis stage 3/4 differential expression data for 5 miRNAs that are differentially expressed in serum samples obtained from patients diagnosed with stage 1 or stage 2 fibrosis and serum samples obtained from patients diagnosed with stage 3 or stage 4 fibrosis. 3 of the miRNAs are decreased in serum samples obtained from patients having a stage 1 or stage 2 fibrosis diagnosis relative to their expression level in serum samples obtained from patients having a stage 3 or stage 4 fibrosis diagnosis. 2 of the miRNAs are increased in serum samples obtained from patients having a stage 1 or stage 2 fibrosis diagnosis relative to their expression level in serum samples obtained from patients having a stage 3 or stage 4 fibrosis diagnosis.

The data presented in Tables 10-14 identifies sets of miRNAs that are differentially expressed in serum samples obtained from patients having different stages of fibrosis and distinguish the presence of a fibrosis disease state from the absence of a fibrosis disease state, and distinguish between less severe (stage 1/2) and more severe (stage 3/4) disease states. The identified miRNAs may be used individually or in combination as biomarkers to identify the fibrosis disease state of a patient based on determining the miRNA expression profile of the selected miRNAs in a serum sample of a patient.

### Example 7: MicroRNA Expression Classifiers for Liver Fibrosis

miR-224 showed strong correlation with liver fibrosis in the data presented in Example 6. A significant modulation of miR-224 in the serum of NAFL patients with fibrosis grades above 0 was identified. Differential expression analysis was done using the R/Bioconductor package limma (Linear Models for Microarray Data). The serum levels were 1.88, 3.01 and 3.42 fold higher in patients with stage 1 liver fibrosis versus no fibrosis, stage 2 vs. no fibrosis and stage 3 & 4 vs. no fibrosis. Therefore, the serum levels of miR-224 correlate with the degree of fibrosis and may be used, alone or in combination with other biomarkers, to monitor liver fibrosis progression.

Serum levels of miR-224 in combination with miR-191 yielded a classifier with the ability to discriminate patients with grade 3 and 4 liver fibrosis vs. no fibrosis with an area under the curve of ˜0.85.

Table 15 lists differentially expressed miRs from Table 12 (Stage 1 vs Stage 0), where the Adjusted P-value is <0.1; Table 16 lists differentially expressed miRs of Table 11 (Stage 2 vs Stage 0), where Adjusted P-value is <0.1; and Table 17 lists differentially expressed miRs from Table 11 (Fibrosis Stage 3 or 4 vs. Stage 0, where the Adjusted P-value is <0.1.

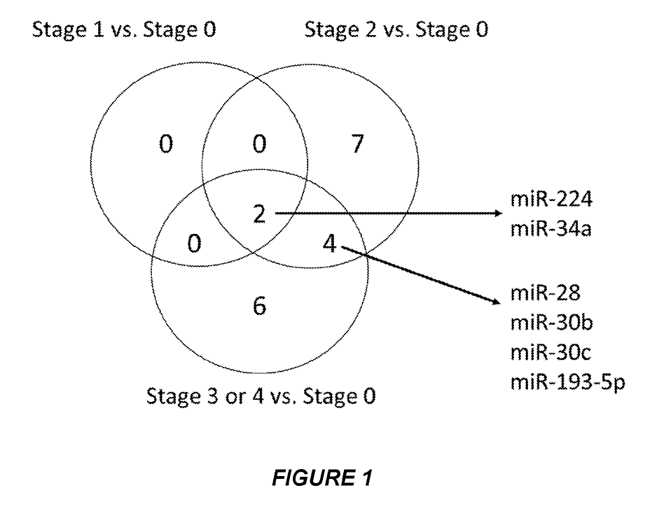

FIG. 1 shows a Venn diagram depicting the number of miRNAs modulated between different stages of fibrosis, relative to abundance of the same miRNAs in the absence of fibrosis. miR-224 and miR-34a were found to be modulated for all fibrosis stages relative to samples without liver fibrosis. miR-28, miR-30b, miR-30c, and miR-193a-5p were found modulated only from samples with liver fibrosis stages 2 and above.

Twelve microRNA Classifier for Liver Fibrosis

The serum microRNA profiles were classified into Advanced Fibrosis (Stages 3 or 4) or No Fibrosis (Stage 0) using the following binary classifiers: Compound Covariate Predictor, Diagonal Linear Discriminant Analysis, and/or Bayesian Compound Covariate Classifier. microRNA selection was done by first identifying microRNAs that were significantly different in a two-sample t-test between the two classes over a range of significance values (0.01, 0.005, 0.001, 0.0005). For each prediction method, the significance value with the lowest cross-validation misclassification rate is chosen to for the predictor. The composition of the 12-microRNA classifier is presented in table 18. The gene weights assigned by each of the three methods are presented in Table 19.

Prediction Rule from the 3 Classification Methods:

The prediction rule is defined by the inner sum of the weights (wi) and expression (xi) of significant genes. The expression is the log ratios for dual-channel data and log intensities for single-channel data.

A sample is classified to the class Advanced Fibrosis if the sum is greater than the threshold; that is,

Σiwixi>threshold

The threshold for the Compound Covariate predictor is 1.683. The threshold for the Diagonal Linear Discriminant predictor is 77.323. The threshold for the Support Vector Machine predictor is 2.268.

Cross-validation was used to test the performance of the classifiers, as follows.

Let, for some class A,

n11=number of class A samples predicted as A,
n12=number of class A samples predicted as non-A,
n21=number of non-A samples predicted as A,
n22=number of non-A samples predicted as non-A.

Then the following parameters can characterize performance of classifiers:

Sensitivity=n11/(n11±n12),

Specificity=n22/(n21+n22),

Positive Predictive Value(PPV)=n11/(n11+n21),

Negative Predictive Value(NPV)=n22/(n12+n22).

Sensitivity is the probability for a class A sample to be correctly predicted as class A. Specificity is the probability for a non class A sample to be correctly predicted as non-A. PPV is the probability that a sample predicted as class A actually belongs to class A. NPV is the probability that a sample predicted as non class A actually does not belong to class A.

The performance of the Compound Covariate Predictor Classifier is presented in Table 20. The performance of the Diagonal Linear Discriminant Analysis Classifier is presented in Table 21. The performance of the Support Vector Machine Classifier is presented in Table 22.

The receiver operator characteristic (ROC) of the classifier was represented graphically. The area under the curve (AUC) obtained averaged 0.81 using 3 classification methods: AUC of 0.82 obtained by Compound Covariate Predictor (CCP), AUC of 0.808 obtained by Diagonal Linear Discriminant Predictor (DLDP) and AUC of 0.803 obtained by Bayesian Compound Covariate Predictor (BCCP).

One Pair (Two microRNA) Classifier for Liver Fibrosis

The serum microRNA profiles were classified into Advanced Fibrosis (Stages 3 or 4) or No Fibrosis (Stage 0) using the following binary classifiers: Compound Covariate Predictor, Diagonal Linear Discriminant Analysis, and/or Support Vector Machines. The number of microRNAs was set to 2 (1 pair). The 1 pair of microRNAs were identified using the greedy-pairs approach (Bo et al. 2002). The greedy-pairs method starts by ranking all microRNAs based on individual t-scores. The best-ranked microRNA is selected, and the procedure then searches for the microRNA that together with the best-ranked microRNA provides the best discrimination and maximizes the pair t-score. The pair is then removed from the set of microRNAs, and the process is repeated on the remaining set of microRNAs until the desired number of pairs of microRNAs is reached. The desired number of pairs is specified a priori. Various numbers of pairs were specified and the one with the best AUC was picked. The notion behind the greedy-pairs method is that methods that would consider each microRNA separately may miss sets of microRNAs that together separate classes well, but not so well individually (Bø et al. 2002).

The composition of the 2-microRNA classifier is presented in table 23. The gene weights assigned by each of the three methods are presented in Table 24.

Prediction Rule from the 3 Classification Methods:

The prediction rule is defined by the inner sum of the weights (wᵢ) and expression (xᵢ) of significant genes. The expression is the log ratios for dual-channel data and log intensities for single-channel data. A sample is classified to the class Advanced Fibrosis if the sum is greater than the threshold; that is,

Σᵢwᵢxᵢ>threshold.

The threshold for the Compound Covariate predictor is −120.631. The threshold for the Diagonal Linear Discriminant predictor is −26.87. The threshold for the Support Vector Machine predictor is −9.785.

Cross-validation was used to test the performance of the classifiers, as follows.

Let, for some class A,

n11=number of class A samples predicted as A,
n12=number of class A samples predicted as non-A,
n21=number of non-A samples predicted as A,
n22=number of non-A samples predicted as non-A.

Then the following parameters can characterize performance of classifiers:

Sensitivity=n11/(n11+n12),

Specificity=n22/(n21+n22),

Positive Predictive Value(PPV)=n11/(n11+n21),

Negative Predictive Value(NPV)=n22/(n12+n22).

Sensitivity is the probability for a class A sample to be correctly predicted as class A. Specificity is the probability for a non class A sample to be correctly predicted as non-A. PPV is the probability that a sample predicted as class A actually belongs to class A. NPV is the probability that a sample predicted as non class A actually does not belong to class A.

The performance of the Compound Covariate Predictor Classifier is presented in Table 25. The performance of the Diagonal Linear Discriminant Analysis Classifier is presented in Table 26. The performance of the Support Vector Machine Classifier is presented in Table 27.

The receiver operator characteristic (ROC) of the classifier was represented graphically. The area under the curve (AUC) obtained averaged 0.85 using 3 classification methods: AUC of 0.855 obtained by Compound Covariate Predictor (CCP), AUC of 0.859 obtained by Diagonal Linear Discriminant Predictor (DLDP) and AUC of 0.842 obtained by Bayesian Compound Covariate Predictor (BCCP).

### Example 8: Identification of MicroRNAs Differentially Expressed in Hepatocellular Ballooning

The 153 samples were classified for hepatocellular ballooning. 33 had stage 0, 86 had stage 1, 28 had stage 2, 1 had stage 3, and 4 had stage 0-1 (counted as score 1 in analysis).

Table 28 presents mean hepatocellular ballooning stage 2/3 vs. hepatocellular ballooning free differential expression data for 29 miRNAs that are differentially expressed in serum samples obtained from patients diagnosed with stage 2 or stage 3 hepatocellular ballooning and serum samples obtained from patients diagnosed as free of hepatocellular ballooning. 17 of the miRNAs are decreased in serum samples obtained from patients having a stage 2 or a stage 3 hepatocellular ballooning diagnosis relative to their expression level in serum samples obtained from patients diagnosed as free of hepatocellular ballooning. 12 of the miRNAs are increased in serum samples obtained from patients having a stage 2 or a stage 3 hepatocellular ballooning diagnosis relative to their expression level in serum samples obtained from patients diagnosed as free of hepatocellular ballooning.

Table 29 presents mean hepatocellular ballooning stage 2/3 vs hepatocellular ballooning stage 1 differential expression data for 20 miRNAs that are differentially expressed in serum samples obtained from patients diagnosed with stage 2 or stage 3 hepatocellular ballooning and serum samples obtained from patients diagnosed with stage 1 hepatocellular ballooning. 6 of the miRNAs are decreased in serum samples obtained from patients having a stage 2 or a stage 3 hepatocellular ballooning diagnosis relative to their expression level in serum samples obtained from patients diagnosed as having a stage 1 hepatocellular ballooning diagnosis. 14 of the miRNAs are increased in serum samples obtained from patients having a stage 2 or a stage 3 hepatocellular ballooning diagnosis relative to their expression level in serum samples obtained from patients diagnosed as having a stage 1 hepatocellular ballooning diagnosis.

The data presented in Tables 28 and 29 identifies sets of miRNAs that are differentially expressed in serum samples obtained from patients having different stages of hepatocellullar ballooning and distinguish the presence of a hepatocellullar ballooning disease state from the absence of a hepatocellullar ballooning disease state, and distinguish between less severe (stage 1/2) and more severe (stage 3) disease states. The identified miRNAs may be used individually or in combination as biomarkers to identify the hepatocellullar ballooning disease state of a patient based on determining the miRNA expression profile of the selected miRNAs in a serum sample of a patient.

### Example 9: MicroRNA Expression Classifiers for Hepatocellular Ballooning

The data presented in Example 8 identify an increase in correlation of miR-224 serum levels with the presence of hepatocellular ballooning. This example describes an eight pair microRNA classifier that discriminates between hepatocellular ballooning scores 2 or 3 and score 0 (NAFL patients without histopathological evidences of HB) and a two pair classifier that discriminates between hepatocellular ballooning scores 2 or 3 and a hepatocellular ballooning score of 1.

8 Pair (16 microRNA) Classifier for Hepatocellular Ballooning

The serum microRNA profiles were classified into Ballooning Score 2 or 3 or Ballooning Score 0 using the following binary classifiers: Compound Covariate Predictor, Diagonal Linear Discriminant Analysis, and/or Support Vector Machines.

The number of microRNAs was set to 16 (8 pairs). These 8 pairs of microRNAs were identified using the greedy-pairs approach (Bo et al. 2002). The greedy-pairs method starts by ranking all microRNAs based on individual t-scores. The best-ranked microRNA is selected, and the procedure then searches for the microRNA that together with the best-ranked microRNA provides the best discrimination and maximizes the pair t-score. The pair is then removed from the set of microRNAs, and the process is repeated on the remaining set of microRNAs until the desired number of pairs of microRNAs is reached. The desired number of pairs is specified a priori. Various numbers of pairs were specified and the one with the best AUC was picked. The notion behind the greedy-pairs method is that methods that would consider each microRNA separately may miss sets of microRNAs that together separate classes well, but not so well individually (Bo et al. 2002).

The composition of the 8 pair classifier is presented in table 30. The gene weights assigned by each of the three methods are presented in Table 31.

Prediction Rule from the 3 Classification Methods:

The prediction rule is defined by the inner sum of the weights (wᵢ) and expression (xᵢ) of significant genes. The expression is the log ratios for dual-channel data and log intensities for single-channel data. A sample is classified to the class Score_0 if the sum is greater than the threshold; that is,

Σᵢwᵢxᵢ>threshold.

The threshold for the Compound Covariate predictor is 401.796. The threshold for the Diagonal Linear Discriminant predictor is 11.023. The threshold for the Support Vector Machine predictor is −43.007.

Cross-validation was used to test the performance of the classifiers, as follows.

Let, for some class A,

n11=number of class A samples predicted as A,
n12=number of class A samples predicted as non-A,
n21=number of non-A samples predicted as A,
n22=number of non-A samples predicted as non-A.

Then the following parameters can characterize performance of classifiers:

Sensitivity=n11/(n11+n12),

Specificity=n22/(n21+n22),

Positive Predictive Value(PPV)=n11/(n11+n21),

Negative Predictive Value(NPV)=n22/(n12+n22).

Sensitivity is the probability for a class A sample to be correctly predicted as class A. Specificity is the probability for a non class A sample to be correctly predicted as non-A. PPV is the probability that a sample predicted as class A actually belongs to class A. NPV is the probability that a sample predicted as non class A actually does not belong to class A.

The performance of the Compound Covariate Predictor Classifier is presented in Table 32. The performance of the Diagonal Linear Discriminant Analysis Classifier is presented in Table 33. The performance of the Support Vector Machine Classifier is presented in Table 34.

The receiver operator characteristic (ROC) of the classifier was represented graphically. The area under the curve (AUC) obtained averaged 0.82 using 3 classification methods: AUC of 0.824 obtained by Compound Covariate Predictor (CCP), AUC of 0.809 obtained by Diagonal Linear Discriminant Predictor (DLDP) and AUC of 0.821 obtained by Bayesian Compound Covariate predictor (BCCP).

Two Pair (4 microRNA) Classifier for Hepatocellular Ballooning

The serum microRNA profiles were classified into Ballooning Score 2 or 3, or Ballooning Score 1 using the following binary classifiers: Compound Covariate Predictor, Diagonal Linear Discriminant Analysis, and/or Support Vector Machines.

The number of microRNAs was set to 4 (2 pairs). These 2 pairs of microRNAs were identified using the greedy-pairs approach (Bo et al. 2002). The greedy-pairs method starts by ranking all microRNAs based on individual t-scores. The best-ranked microRNA is selected, and the procedure then searches for the microRNA that together with the best-ranked microRNA provides the best discrimination and maximizes the pair t-score. The pair is then removed from the set of microRNAs, and the process is repeated on the remaining set of microRNAs until the desired number of pairs of microRNAs is reached. The desired number of pairs is specified a priori. Various numbers of pairs were specified and the one with the best AUC was picked. The notion behind the greedy-pairs method is that methods that would consider each microRNA separately may miss sets of microRNAs that together separate classes well, but not so well individually (Bo et al. 2002).

The composition of the 2 pair classifier is presented in table 35. The gene weights assigned by each of the three methods are presented in Table 36.

Prediction Rule from the 3 Classification Methods:

The prediction rule is defined by the inner sum of the weights (wᵢ) and expression (xᵢ) of significant genes. The expression is the log ratios for dual-channel data and log intensities for single-channel data. A sample is classified to the class Score_1 if the sum is greater than the threshold; that is,

Σᵢwᵢxᵢ>threshold.

The threshold for the Compound Covariate predictor is 71.576. The threshold for the Diagonal Linear Discriminant predictor is −8.12. The threshold for the Support Vector Machine predictor is −5.262.

Cross-validation was used to test the performance of the classifiers, as follows.

Let, for some class A,

n11=number of class A samples predicted as A,
n12=number of class A samples predicted as non-A,
n21=number of non-A samples predicted as A,
n22=number of non-A samples predicted as non-A.

Then the following parameters can characterize performance of classifiers:

Sensitivity=n11/(n11+n12),

Specificity=n22/(n21+n22),

Positive Predictive Value(PPV)=n11/(n11+n21),

Negative Predictive Value(NPV)=n22/(n12+n22).

Sensitivity is the probability for a class A sample to be correctly predicted as class A. Specificity is the probability for a non class A sample to be correctly predicted as non-A. PPV is the probability that a sample predicted as class A actually belongs to class A. NPV is the probability that a sample predicted as non class A actually does not belong to class A.

The performance of the Compound Covariate Predictor Classifier is presented in Table 37. The performance of the Diagonal Linear Discriminant Analysis Classifier is presented in Table 38. The performance of the Support Vector Machine Classifier is presented in Table 39.

The receiver operator characteristic (ROC) of the classifier was represented graphically. The area under the curve (AUC) obtained averaged 0.76 using 3 classification methods: AUC of 0.77 obtained by Compound Covariate Predictor (CCP), AUC of 0.757 obtained by Diagonal Linear Discriminant Predictor (DLDP) and AUC of 0.754 obtained by Bayesian Compound Covariate Predictor (BCCP).

TABLE 1

| | | Linear | | | adj.P. | | SEQ ID |
|---|---|---|---|---|---|---|---|
| ID | logFC | FC | AveExpr | P.Value | Val | miR_Sequence | NO: |
| 000439_hsa-miR-103_A | -1.34 | 0.39 | 25.60 | 0.0084 | 0.0968 | AGCAGCAUUGUACAGGGCUAUGA | 1 |
| 002257_hsa-miR-339-5p_A | -0.93 | 0.53 | 26.49 | 0.0210 | 0.1731 | UCCCUGUCCUCCAGGAGCUCACG | 2 |
| 001319_mmu-miR-374- | -0.87 | 0.55 | 23.35 | 0.0455 | 0.2385 | AUAUAAUACAACCUGCUAAGUG | 3 |
| 5p_A | | | | | | | |
| 002278_hsa-miR-145_A | -0.67 | 0.63 | 26.54 | 0.0131 | 0.1248 | GUCCAGUUUUCCCAGGAAUCCCU | 4 |
| 001986_hsa-miR-766_B | -0.51 | 0.70 | 23.65 | 0.0394 | 0.2289 | ACUCCAGCCCCACAGCCUCAGC | 5 |
| 001562_hsa-miR-629_B | -0.51 | 0.70 | 27.26 | 0.0053 | 0.0733 | GU UCUCCCAACGUAAGCCCAGC | 6 |
| 002299_hsa-miR-191_A | -0.47 | 0.72 | 18.60 | 0.0110 | 0.1193 | CAACGGAAUCCCAAAAGCAGCUG | 7 |
| 000565_hsa-miR-376a_A | -0.43 | 0.74 | 22.95 | 0.0324 | 0.2109 | AUCAUAGAGGAAAAUCCACGU | 8 |
| 000411_hsa-miR-28_A | -0.43 | 0.74 | 23.24 | 0.0013 | 0.0367 | AAGGAGCUCACAGUCUAUUGAG | 9 |
| 000528_hsa-miR-301_A | -0.40 | 0.76 | 23.88 | 0.0041 | 0.0702 | CAGUGCAAUAGUAUUGUCAAAGC | 10 |
| 002283_hsa-let-7d_A | -0.40 | 0.76 | 25.07 | 0.0059 | 0.0733 | AGAGGUAGUAGGUUGCAUAGUU | 11 |
| 000419_hsa-miR-30c_A | -0.40 | 0.76 | 18.26 | 2.9846E- | 0.0052 | UGUAAACAUCCUACACUCUCAGC | 12 |
| | | | | 05 | | | |
| 000602_hsa-miR-30b_A | -0.35 | 0.78 | 18.16 | 0.0013 | 0.0367 | UGUAAACAUCCUACACUCAGCU | 13 |
| 002422_hsa-miR-18a_A | -0.32 | 0.80 | 24.91 | 0.0329 | 0.2109 | UAAGGUGCAUCUAGUGCAGAUAG | 14 |
| 001286_hsa-miR-539_A | -0.31 | 0.80 | 27.70 | 0.0053 | 0.0733 | GGAGAAAUUAUCCUUGGUGUGU | 15 |
| 000524_hsa-miR-221_A | -0.30 | 0.81 | 20.62 | 0.0144 | 0.1248 | AGCUACAUUGUCUGCUGGGUUUC | 16 |
| 002259_hsa-miR-340- | -0.30 | 0.81 | 27.15 | 0.0438 | 0.2370 | UCCGUCUCAGUUACUUUAUAGC | 17 |
| star_B | | | | | | | |
| 000436_hsa-miR-99b_A | -0.29 | 0.82 | 22.50 | 0.0264 | 0.1982 | CACCCGUAGAACCGACCUUGCG | 18 |
| 000545_hsa-miR-331_A | -0.29 | 0.82 | 20.99 | 0.0018 | 0.0380 | GCCCCUGGGCCUAUCCUAGAA | 19 |
| 002198_hsa-miR-125a- | -0.29 | 0.82 | 27.62 | 0.0437 | 0.2370 | UCCCUGAGACCCUUUAACCUGUGA | 20 |
| 5p_A | | | | | | | |
| 002228_hsa-miR-126_A | -0.21 | 0.87 | 17.93 | 0.0397 | 0.2289 | UCGUACCGUGAGUAAUAAUGCG | 21 |
| 000543_hsa-miR-328_A | -0.19 | 0.87 | 20.30 | 0.0297 | 0.2065 | CUGGCCCUCUCUGCCCUUCCGU | 22 |
| 001285_hsa-miR-487b_A | -0.14 | 0.91 | 27.84 | 0.0347 | 0.2145 | AAUCGUACAGGGUCAUCCACUU | 23 |
| 000420_hsa-miR-30d_B | 0.23 | 1.17 | 20.46 | 0.0059 | 0.0733 | UGUAAACAUCCCCGACUGGAAG | 24 |
| 000417_hsa-miR-30a-5p_B | 0.27 | 1.21 | 17.97 | 0.0006 | 0.0254 | UGUAAACAUCCUCGACUGGAAG | 25 |
| 000475_hsa-miR-152_A | 0.28 | 1.21 | 22.71 | 0.0141 | 0.1248 | UCAGUGCAUGACAGAACUUGG | 26 |
| 001515_hsa-miR-660_A | 0.31 | 1.24 | 21.83 | 0.0121 | 0.1236 | UACCCAUUGCAUAUCGGAGUUG | 27 |
| 000491_hsa-miR-192_A | 0.50 | 1.42 | 19.93 | 0.0249 | 0.1960 | CUGACCUAUGAAUUGACAGCC | 28 |
| 002367_hsa-miR-193b_A | 0.60 | 1.51 | 20.83 | 0.0298 | 0.2065 | AACUGGCCCUCAAAGUCCCGCU | 29 |
| 002089_hsa-miR-505_A | 0.60 | 1.52 | 27.08 | 0.0002 | 0.0134 | CGUCAACACUUGCUGGUUUCCU | 30 |
| 002281_hsa-miR-193a- | 0.61 | 1.53 | 23.76 | 0.0015 | 0.0367 | UGGGUCUUUGCGGGCGAGAUGA | 31 |
| 5p_A | | | | | | | |
| 002099_hsa-miR-224_A | 0.77 | 1.70 | 25.98 | 0.0038 | 0.0702 | CAAGUCACUAGUGGUUCCGUU | 32 |
| 000426_hsa-miR-34a_A | 1.07 | 2.10 | 23.56 | 0.0002 | 0.0134 | UGGCAGUGUCUUAGCUGGUUGU | 33 |

TABLE 2

| | | Linear | | | adj.P. | | SEQ ID |
|---|---|---|---|---|---|---|---|
| ID | logFC | FC | AveExpr | P.Value | Val | miR_Sequence | NO: |
| 000439_hsa-miR-103_A | -1.87 | 0.27 | 25.60 | 0.0053 | 0.1983 | AGCAGCAUUGUACAGGGCUAUGA | 34 |
| 002278_hsa-miR-145_A | -0.76 | 0.59 | 26.54 | 0.0331 | 0.2933 | GUCCAGUUUUCCCAGGAAUCCCU | 35 |
| 002352_hsa-miR-652_A | -0.65 | 0.64 | 25.85 | 0.0222 | 0.2933 | AAUGGCGCCACUAGGGUUGUG | 36 |
| 000411_hsa-miR-28_A | -0.59 | 0.67 | 23.24 | 0.0008 | 0.1377 | AAGGAGCUCACAGUCUAUUGAG | 37 |
| 000544_hsa-miR-330_A | -0.58 | 0.67 | 27.03 | 0.0160 | 0.2739 | GCAAAGCACACGGCCUGCAGAGA | 38 |
| 002299_hsa-miR-191_A | -0.55 | 0.69 | 18.60 | 0.0247 | 0.2933 | CAACGGAAUCCCAAAAGCAGCUG | 39 |
| 000528_hsa-miR-301_A | -0.49 | 0.71 | 23.88 | 0.0076 | 0.2180 | CAGUGCAAUAGUAUUGUCAAAGC | 40 |
| 002259_hsa-miR-340-star_B | -0.47 | 0.72 | 27.15 | 0.0171 | 0.2739 | UCCGUCUCAGUUACUUUAUAGC | 41 |
| 002295_hsa-miR-223_A | -0.40 | 0.76 | 13.30 | 0.0447 | 0.3365 | UGUCAGUUUGUCAAAUACCCCA | 42 |
| 002285_hsa-miR-186_A | -0.37 | 0.77 | 22.16 | 0.0143 | 0.2739 | CAAAGAAUUCUCCUUUUGGGCU | 43 |
| 000419_hsa-miR-30c_A | -0.37 | 0.77 | 18.26 | 0.0029 | 0.1980 | UGUAAACAUCCUACACUCUCAGC | 44 |
| 000524_hsa-miR-221_A | -0.35 | 0.78 | 20.62 | 0.0301 | 0.2933 | AGCUACAUUGUCUGCUGGGUUUC | 45 |
| 000602_hsa-miR-30b_A | -0.31 | 0.81 | 18.16 | 0.0339 | 0.2933 | UGUAAACAUCCUACACUCAGCU | 46 |
| 002642_HSA-MIR-151-5P_B | -0.29 | 0.82 | 27.85 | 0.0034 | 0.1980 | UCGAGGAGCUCACAGUCUAGU | 47 |
| 000545_hsa-miR-331_A | -0.25 | 0.84 | 20.99 | 0.0371 | 0.3058 | GCCCCUGGGCCUAUCCUAGAA | 48 |
| 000543_hsa-miR-328_A | -0.24 | 0.85 | 20.30 | 0.0390 | 0.3069 | CUGGCCCUCUCUGCCCUUCCGU | 49 |
| 002317_hsa-miR-181a-2- | -0.23 | 0.85 | 27.83 | 0.0279 | 0.2933 | ACCACUGACCGUUGACUGUACC | 50 |
| star_B | | | | | | | |
| 002277_hsa-miR-320_A | 0.34 | 1.26 | 18.10 | 0.0338 | 0.2933 | AAAAGCUGGGUUGAGAGGGCGA | 51 |
| 001515_hsa-miR-660_A | 0.39 | 1.31 | 21.83 | 0.0141 | 0.2739 | UACCCAUUGCAUAUCGGAGUUG | 52 |
| 002089_hsa-miR-505_A | 0.41 | 1.33 | 27.08 | 0.0497 | 0.3428 | CGUCAACACUUGCUGGUUUCCU | 53 |
| 002844_HSA-MIR-320B_B | 0.46 | 1.38 | 25.72 | 0.0174 | 0.2739 | AAAAGCUGGGUUGAGAGGGCAA | 54 |
| 000433_hsa-miR-95_A | 0.51 | 1.43 | 26.93 | 0.0307 | 0.2933 | UUCAACGGGUAUUUAUUGAGCA | 55 |
| 000491_hsa-miR-192_A | 0.63 | 1.55 | 19.93 | 0.0309 | 0.2933 | CUGACCUAUGAAUUGACAGCC | 56 |
| 000426_hsa-miR-34a_A | 1.05 | 2.07 | 23.56 | 0.0057 | 0.1983 | UGGCAGUGUCUUAGCUGGUUGU | 57 |

TABLE 3

| | | Linear | | | adj.P. | | SEQ ID |
|---|---|---|---|---|---|---|---|
| ID | logFC | FC | AveExpr | P.Value | Val | miR_Sequence | NO: |
| 001562_hsa-miR-629_B | -0.67 | 0.63 | 27.26 | 0.0064 | 0.1231 | GUUCUCCCAACGUAAGCCCAGC | 58 |
| 000436_hsa-miR-99b_A | -0.53 | 0.69 | 22.50 | 0.0027 | 0.0930 | CACCCGUAGAACCGACCUUGCG | 59 |
| 002283_hsa-let-7d_A | -0.44 | 0.74 | 25.07 | 0.0242 | 0.2984 | AGAGGUAGUAGGUUGCAUAGUU | 60 |
| 000419_hsa-miR-30c_A | -0.43 | 0.74 | 18.26 | 0.0008 | 0.0433 | UGUAAACAUCCUACACUCUCAGC | 61 |
| 000602_hsa-miR-30b_A | -0.40 | 0.76 | 18.16 | 0.0064 | 0.1231 | UGUAAACAUCCUACACUCAGCU | 62 |
| 001286_hsa-miR-539_A | -0.35 | 0.78 | 27.70 | 0.0192 | 0.2551 | GGAGAAAUUAUCCUUGGUGUGU | 63 |
| 000545_hsa-miR-331_A | -0.33 | 0.80 | 20.99 | 0.0080 | 0.1379 | GCCCCUGGGCCUAUCCUAGAA | 64 |
| 002289_hsa-miR-139-5p_A | -0.29 | 0.82 | 21.92 | 0.0451 | 0.4585 | UCUACAGUGCACGUGUCUCCAG | 65 |
| 001285_hsa-miR-487b_A | -0.22 | 0.86 | 27.84 | 0.0142 | 0.2053 | AAUCGUACAGGGUCAUCCACUU | 66 |
| 000420_hsa-miR-30d_B | 0.37 | 1.29 | 20.46 | 0.0012 | 0.0519 | UGUAAACAUCCCCGACUGGAAG | 67 |
| 000417_hsa-miR-30a-5p_B | 0.39 | 1.31 | 17.97 | 0.0002 | 0.0217 | UGUAAACAUCCUCGACUGGAAG | 68 |
| 001984_hsa-miR-590-5p_A | 0.43 | 1.35 | 22.37 | 0.0360 | 0.4149 | GAGCUUAUUCAUAAAAGUGCAG | 69 |
| 002245_hsa-miR-122_A | 0.69 | 1.61 | 19.47 | 0.0384 | 0.4149 | UGGAGUGUGACAAUGGUGUUUG | 70 |
| 002281_hsa-miR-193a-5p_A | 0.75 | 1.69 | 23.76 | 0.0035 | 0.1014 | UGGGUCUUUGCGGGCGAGAUGA | 71 |
| 002089_hsa-miR-505_A | 0.80 | 1.74 | 27.08 | 0.0003 | 0.0217 | CGUCAACACUUGCUGGUUUCCU | 72 |
| 002099_hsa-miR-224_A | 0.92 | 1.90 | 25.98 | 0.0098 | 0.1545 | CAAGUCACUAGUGGUUCCGUU | 73 |
| 000426_hsa-miR-34a_A | 1.10 | 2.14 | 23.56 | 0.0049 | 0.1206 | UGGCAGUGUCUUAGCUGGUUGU | 74 |

TABLE 4

| | | Linear | | | adj.P. | | SEQ ID |
|---|---|---|---|---|---|---|---|
| ID | logFC | FC | AveExpr | P.Value | Val | miR_Sequence | NO: |
| 002352_hsa-miR-652_A | -0.97 | 0.51 | 25.85 | 0.0112 | 0.4715 | AAUGGCGCCACUAGGGUUGUG | 75 |
| 000413_hsa-miR-29b_A | -0.65 | 0.64 | 27.30 | 0.0152 | 0.4715 | UAGCACCAUUUGAAAUCAGUGUU | 76 |
| 002285_hsa-miR-186_A | -0.47 | 0.72 | 22.16 | 0.0207 | 0.5106 | CAAAGAAUUCUCCUUUUGGGCU | 77 |
| 002642_HSA-MIR-151-5P_B | -0.40 | 0.76 | 27.85 | 0.0028 | 0.4715 | UCGAGGAGCUCACAGUCUAGU | 78 |
| 002317_hsa-miR-181a-2-star_B | -0.30 | 0.81 | 27.83 | 0.0301 | 0.5779 | ACCACUGACCGUUGACUGUACC | 79 |
| 000436_hsa-miR-99b_A | 0.46 | 1.38 | 22.50 | 0.0427 | 0.7381 | CACCCGUAGAACCGACCUUGCG | 80 |
| 002277_hsa-miR-320_A | 0.47 | 1.39 | 18.10 | 0.0257 | 0.5566 | AAAAGCUGGGUUGAGAGGGCGA | 81 |
| 002844_HSA-MIR-320B_B | 0.63 | 1.54 | 25.72 | 0.0164 | 0.4715 | AAAAGCUGGGUUGAGAGGGCAA | 82 |
| 000433_hsa-miR-95_A | 0.79 | 1.73 | 26.93 | 0.0125 | 0.4715 | UUCAACGGGUAUUUAUUGAGCA | 83 |
| 002243_hsa-miR-378_B | 1.95 | 3.86 | 26.68 | 0.0157 | 0.4715 | ACUGGACUUGGAGUCAGAAGG | 84 |

TABLE 5

| | | | | Geom mean | Geom mean | | |
|---|---|---|---|---|---|---|---|
| | | Parametric | | of intensities | of intensities | Fold- | |
| | Pair | p-value | t-value | in class 1 | in class 2 | change | UniqueID |
| 1 | 1 | 2.47E−05 | −4.356 | 17.96 | 18.36 | 0.76 | 000419_hsa-miR-30c_A |
| 2 | 1 | 0.0002536 | 3.75 | 24.38 | 23.31 | 2.1 | 000426_hsa-miR-34a_A |
| 3 | 2 | 0.0002359 | 3.77 | 27.54 | 26.94 | 1.52 | 002089_hsa-miR-505_A |
| 4 | 2 | 0.0040421 | −2.921 | 23.57 | 23.97 | 0.76 | 000528_hsa-miR-301_A |
| 5 | 3 | 0.0004607 | 3.583 | 18.18 | 17.91 | 1.21 | 000417_hsa-miR-30a-5p_B |
| 6 | 3 | 0.0054114 | −2.823 | 26.87 | 27.38 | 0.7 | 001562_hsa-miR-629_B |
| 7 | 4 | 0.0012378 | −3.294 | 17.89 | 18.25 | 0.78 | 000602_hsa-miR-30b_A |
| 8 | 4 | 0.0136399 | −2.497 | 26.02 | 26.69 | 0.63 | 002278_hsa-miR-145_A |
| 9 | 5 | 0.0012413 | −3.293 | 22.91 | 23.34 | 0.74 | 000411_hsa-miR-28_A |
| 10 | 5 | 0.0136525 | 2.497 | 22.92 | 22.64 | 1.21 | 000475_hsa-miR-152_A |
| 11 | 6 | 0.0015432 | 3.227 | 24.23 | 23.62 | 1.53 | 002281_hsa-miR-193a-5p_A |
| 12 | 6 | 0.0051988 | 2.837 | 20.63 | 20.40 | 1.17 | 000420_hsa-miR-30d_B |
| 13 | 7 | 0.0015552 | −3.224 | 20.77 | 21.06 | 0.82 | 000545_hsa-miR-331_A |
| 14 | 7 | 0.0040454 | 2.921 | 26.56 | 25.80 | 1.7 | 002099_hsa-miR-224_A |
| 15 | 8 | 0.005055 | −2.846 | 27.46 | 27.78 | 0.8 | 001286_hsa-miR-539_A |
| 16 | 8 | 0.0329974 | −2.152 | 24.67 | 24.98 | 0.8 | 002422_hsa-miR-18a_A |
| 17 | 9 | 0.005923 | −2.793 | 24.76 | 25.16 | 0.76 | 002283_hsa-let-7d_A |
| 18 | 9 | 0.0112904 | −2.566 | 18.24 | 18.71 | 0.72 | 002299_hsa-miR-191_A |
| 19 | 10 | 0.0088822 | −2.652 | 24.57 | 25.92 | 0.39 | 000439_hsa-miR-103_A |
| 20 | 10 | 0.0886247 | 1.714 | 27.67 | 27.33 | 1.27 | 001592_hsa-miR-642_A |

TABLE 6

| | | | Diagonal | |
|---|---|---|---|---|
| | | Compound | Linear | Support |
| | | Covariate | Discriminant | Vector |
| | Genes | Predictor | Analysis | Machines |
| 1 | 000411_hsa-miR-28_A | −3.2931 | −0.9428 | 0.418 |
| 2 | 000419_hsa-miR-30c_A | −4.3564 | −1.7781 | −1.0184 |
| 3 | 000426_hsa-miR-34a_A | 3.7501 | 0.4895 | 0.2266 |
| 4 | 000439_hsa-miR-103_A | −2.6519 | −0.1954 | −0.0873 |
| 5 | 000475_hsa-miR-152_A | 2.4965 | 0.8395 | 0.1828 |
| 6 | 000528_hsa-miR-301_A | −2.9208 | −0.792 | −0.3502 |
| 7 | 000545_hsa-miR-331_A | −3.2245 | −1.3415 | 0.4874 |
| 8 | 000602_hsa-miR-30b_A | −3.2944 | −1.1785 | 0.1516 |
| 9 | 001286_hsa-miR-539_A | −2.8463 | −0.9641 | −0.2186 |
| 10 | 001592_hsa-miR-642_A | 1.7141 | 0.3217 | 0.5188 |
| 11 | 002089_hsa-miR-505_A | 3.7699 | 0.8816 | 0.346 |
| 12 | 002099_hsa-miR-224_A | 2.9206 | 0.4143 | 0.2344 |
| 13 | 002278_hsa-miR-145_A | −2.4967 | −0.3457 | −0.2011 |
| 14 | 002281_hsa-miR-193a- | 3.2268 | 0.6358 | 0.129 |
| | 5p_A | | | |
| 15 | 002283_hsa-let-7d_A | −2.7927 | −0.7245 | 0.2348 |
| 16 | 002299_hsa-miR-191_A | −2.5659 | −0.5228 | −0.4328 |
| 17 | 002422_hsa-miR-18a_A | −2.1524 | −0.5465 | 0.0092 |
| 18 | 000417_hsa-miR-30a- | 3.5833 | 1.7607 | −0.039 |
| | 5p_B | | | |
| 19 | 000420_hsa-miR-30d_B | 2.8369 | 1.295 | 0.3895 |
| 20 | 001562_hsa-miR-629_B | −2.8234 | −0.5826 | −0.1822 |

TABLE 7

| Class | Sensitivity | Specificity | PPV | NPV |
|---|---|---|---|---|
| NAFLD | 0.571 | 0.632 | 0.323 | 0.828 |
| NASH | 0.632 | 0.571 | 0.828 | 0.323 |

TABLE 8

| Class | Sensitivity | Specificity | PPV | NPV |
|---|---|---|---|---|
| NAFLD | 0.629 | 0.632 | 0.344 | 0.847 |
| NASH | 0.632 | 0.629 | 0.847 | 0.344 |

TABLE 9

| Class | Sensitivity | Specificity | PPV | NPV |
|---|---|---|---|---|
| NAFLD | 0.229 | 0.86 | 0.333 | 0.784 |
| NASH | 0.86 | 0.229 | 0.784 | 0.333 |

TABLE 10

| | | Linear | | | | | SEQ ID |
|---|---|---|---|---|---|---|---|
| ID | logFC | FC | AveExpr | P.Value | adj.P.Val | miR_Sequence | NO: |
| 000439_hsa-miR-103_A | -1.53 | 0.35 | 25.60 | 0.0210 | 0.1980 | AGCAGCAUUGUACAGGGCUAUGA | 85 |
| 002257_hsa-miR-339- | -1.34 | 0.40 | 26.49 | 0.0093 | 0.1072 | UCCCUGUCCUCCAGGAGCUCACG | 86 |
| 5p_A | | | | | | | |
| 000411_hsa-miR-28_A | -0.68 | 0.62 | 23.24 | 5.8745E-05 | 0.003387628 | AAGGAGCUCACAGUCUAUUGAG | 87 |
| 002299_hsa-miR-191_A | -0.68 | 0.62 | 18.60 | 0.0044 | 0.0696 | CAACGGAAUCCCAAAAGCAGCUG | 88 |
| 002122_hsa-miR-376c_A | -0.66 | 0.63 | 24.09 | 0.0267 | 0.1980 | AACAUAGAGGAAAUUCCACGU | 89 |
| 000565_hsa-miR-376a_A | -0.60 | 0.66 | 22.95 | 0.0170 | 0.1728 | AUCAUAGAGGAAAAUCCACGU | 90 |
| 002422_hsa-miR-18a_A | -0.55 | 0.68 | 24.91 | 0.0032 | 0.0689 | UAAGGUGCAUCUAGUGCAGAUAG | 91 |
| 000436_hsa-miR-99b_A | -0.55 | 0.68 | 22.50 | 0.0010 | 0.0297 | CACCCGUAGAACCGACCUUGCG | 92 |
| 002198_hsa-miR-125a- | -0.47 | 0.72 | 27.62 | 0.0090 | 0.1072 | UCCCUGAGACCCUUUAACCUGUGA | 93 |
| 5p_A | | | | | | | |
| 000419_hsa-miR-30c_A | -0.46 | 0.73 | 18.26 | 0.0002 | 0.0081 | UGUAAACAUCCUACACUCUCAGC | 94 |
| 000602_hsa-miR-30b_A | -0.44 | 0.74 | 18.16 | 0.0015 | 0.0362 | UGUAAACAUCCUACACUCAGCU | 95 |
| 002283_hsa-let-7d_A | -0.40 | 0.76 | 25.07 | 0.0326 | 0.2170 | AGAGGUAGUAGGUUGCAUAGUU | 96 |
| 002259_hsa-miR-340- | -0.39 | 0.76 | 27.15 | 0.0457 | 0.2824 | UCCGUCUCAGUUACUUUAUAGC | 97 |
| star_B | | | | | | | |
| 000545_hsa-miR-331_A | -0.31 | 0.80 | 20.99 | 0.0090 | 0.1072 | GCCCCUGGGCCUAUCCUAGAA | 98 |
| 000543_hsa-miR-328_A | -0.30 | 0.81 | 20.30 | 0.0100 | 0.1086 | CUGGCCCUCUCUGCCCUUCCGU | 99 |
| 000417_hsa-miR-30a- | 0.22 | 1.17 | 17.97 | 0.0302 | 0.2093 | UGUAAACAUCCUCGACUGGAAG | 100 |
| 5p_B | | | | | | | |
| 000433_hsa-miR-95_A | 0.50 | 1.41 | 26.93 | 0.0344 | 0.2207 | UUCAACGGGUAUUUAUUGAGCA | 101 |
| 002089_hsa-miR-505_A | 0.62 | 1.53 | 27.08 | 0.0037 | 0.0696 | CGUCAACACUUGCUGGUUUCCU | 102 |
| 000449_hsa-miR-125b_A | 0.62 | 1.53 | 24.54 | 0.0231 | 0.1980 | UCCCUGAGACCCUAACUUGUGA | 103 |
| 000491_hsa-miR-192_A | 0.63 | 1.55 | 19.93 | 0.0270 | 0.1980 | CUGACCUAUGAAUUGACAGCC | 104 |
| 002296_hsa-miR-885- | 0.68 | 1.60 | 20.38 | 0.0257 | 0.1980 | UCCAUUACACUACCCUGCCUCU | 105 |
| 5p_A | | | | | | | |
| 000521_hsa-miR-218_A | 0.73 | 1.66 | 26.35 | 0.0049 | 0.0703 | UUGUGCUUGAUCUAACCAUGU | 106 |
| 002367_hsa-miR-193b_A | 0.78 | 1.72 | 20.83 | 0.0252 | 0.1980 | AACUGGCCCUCAAAGUCCCGCU | 107 |
| 000564_hsa-miR-375_A | 0.79 | 1.73 | 22.45 | 0.0041 | 0.0696 | UUUGUUCGUUCGGCUCGCGUGA | 108 |
| 002281_hsa-miR-193a- | 0.83 | 1.78 | 23.76 | 0.0006 | 0.0215 | UGGGUCUUUGCGGGCGAGAUGA | 109 |
| 5p_A | | | | | | | |
| 000426_hsa-miR-34a_A | 1.51 | 2.85 | 23.56 | 3.16685E- | 0.002739328 | UGGCAGUGUCUUAGCUGGUUGU | 110 |
| | | | | 05 | | | |
| 002099_hsa-miR-224_A | 1.77 | 3.42 | 25.98 | 3.58858E- | 6.20825E-06 | CAAGUCACUAGUGGUUCCGUU | 111 |
| | | | | 08 | | | |
| 001558_hsa-miR-601_B | 2.25 | 4.76 | 26.13 | 0.0275 | 0.1980 | UGGUCUAGGAUUGUUGGAGGAG | 112 |

TABLE 11

| | | Linear | | | | | SEQ ID |
|---|---|---|---|---|---|---|---|
| ID | logFC | FC | AveExpr | P.Value | adj.P.Val | miR_Sequence | NO: |
| 002257_hsa-miR-339- | -1.41 | 0.38 | 26.49 | 0.0148 | 0.1389 | UCCCUGUCCUCCAGGAGCUCACG | 113 |
| 5p_A | | | | | | | |
| 002323_hsa-miR-454_A | -1.18 | 0.44 | 25.66 | 0.0340 | 0.2180 | UAGUGCAAUAUUGCUUAUAGGGU | 114 |
| 000565_hsa-miR-376a_A | -0.96 | 0.51 | 22.95 | 0.0008 | 0.0292 | AUCAUAGAGGAAAAUCCACGU | 115 |
| 001097_hsa-miR-146b_A | -0.71 | 0.61 | 22.17 | 0.0002 | 0.0141 | UGAGAACUGAAUUCCAUAGGCU | 116 |
| 002283_hsa-let-7d_A | -0.59 | 0.66 | 25.07 | 0.0053 | 0.0824 | AGAGGUAGUAGGUUGCAUAGUU | 117 |
| 002422_hsa-miR-18a_A | -0.55 | 0.68 | 24.91 | 0.0095 | 0.1025 | UAAGGUGCAUCUAGUGCAGAUAG | 118 |
| 000411_hsa-miR-28_A | -0.54 | 0.69 | 23.24 | 0.0039 | 0.0824 | AAGGAGCUCACAGUCUAUUGAG | 119 |
| 000602_hsa-miR-30b_A | -0.53 | 0.69 | 18.16 | 0.0007 | 0.0292 | UGUAAACAUCCUACACUCAGCU | 120 |
| 002355_hsa-miR-532- | -0.51 | 0.70 | 26.53 | 0.0153 | 0.1389 | CCUCCCACACCCAAGGCUUGCA | 121 |
| 3p_A | | | | | | | |
| 002324_hsa-miR-744_A | -0.46 | 0.73 | 24.73 | 0.0353 | 0.2180 | UGCGGGGCUAGGGCUAACAGCA | 122 |
| 000419_hsa-miR-30c_A | -0.37 | 0.77 | 18.26 | 0.0065 | 0.0863 | UGUAAACAUCCUACACUCUCAGC | 123 |
| 000524_hsa-miR-221_A | -0.36 | 0.78 | 20.62 | 0.0456 | 0.2689 | AGCUACAUUGUCUGCUGGGUUUC | 124 |
| 000468_hsa-miR-146a_A | -0.36 | 0.78 | 17.44 | 0.0335 | 0.2180 | UGAGAACUGAAUUCCAUGGGUU | 125 |
| 001138_mmu-miR-379_A | -0.35 | 0.78 | 27.64 | 0.0466 | 0.2689 | UGGUAGACUAUGGAACGUAGG | 126 |
| 002228_hsa-miR-126_A | -0.34 | 0.79 | 17.93 | 0.0177 | 0.1533 | UCGUACCGUGAGUAAUAAUGCG | 127 |
| 002277_hsa-miR-320_A | 0.38 | 1.30 | 18.10 | 0.0321 | 0.2180 | AAAAGCUGGGUUGAGAGGGCGA | 128 |
| 000475_hsa-miR-152_A | 0.46 | 1.37 | 22.71 | 0.0056 | 0.0824 | UCAGUGCAUGACAGAACUUGG | 129 |
| 001551_hsa-miR-597_A | 0.52 | 1.43 | 27.44 | 0.0234 | 0.1892 | UGUGUCACUCGAUGACCACUGU | 130 |
| 002432_hsa-miR-625- | 0.56 | 1.47 | 27.50 | 0.0343 | 0.2180 | GACUAUAGAACUUUCCCCCUCA | 131 |
| star_B | | | | | | | |
| 002245_hsa-miR-122_A | 0.78 | 1.71 | 19.47 | 0.0307 | 0.2180 | UGGAGUGUGACAAUGGUGUUUG | 132 |
| 001020_hsa-miR-365_A | 0.78 | 1.72 | 27.46 | 0.0093 | 0.1025 | UAAUGCCCCUAAAAAUCCUUAU | 133 |
| 002338_hsa-miR-483- | 0.79 | 1.73 | 21.10 | 0.0057 | 0.0824 | AAGACGGGAGGAAAGAAGGGAG | 134 |
| 5p_A | | | | | | | |
| 000491_hsa-miR-192_A | 0.80 | 1.74 | 19.93 | 0.0131 | 0.1335 | CUGACCUAUGAAUUGACAGCC | 135 |
| 002281_hsa-miR-193a- | 0.88 | 1.84 | 23.76 | 0.0013 | 0.0385 | UGGGUCUUUGCGGGCGAGAUGA | 136 |
| 5p_A | | | | | | | |
| 002296_hsa-miR-885- | 0.97 | 1.96 | 20.38 | 0.0046 | 0.0824 | UCCAUUACACUACCCUGCCUCU | 137 |
| 5p_A | | | | | | | |
| 000515_hsa-miR-212_A | 1.00 | 1.99 | 27.28 | 0.0089 | 0.1025 | UAACAGUCUCCAGUCACGGCC | 138 |
| 002367_hsa-miR-193b_A | 1.18 | 2.26 | 20.83 | 0.0029 | 0.0718 | AACUGGCCCUCAAAGUCCCGCU | 139 |
| 002260_hsa-miR-342- | 1.47 | 2.77 | 26.65 | 0.0241 | 0.1892 | UCUCACACAGAAAUCGCACCCGU | 140 |
| 3p_A | | | | | | | |
| 000426_hsa-miR-34a_A | 1.56 | 2.96 | 23.56 | 0.0001 | 0.0108 | UGGCAGUGUCUUAGCUGGUUGU | 141 |
| 002099_hsa-miR-224_A | 1.59 | 3.01 | 25.98 | 8.27984E-06 | 0.001432413 | CAAGUCACUAGUGGUUCCGUU | 142 |

TABLE 12

| ID | logFC | Linear FC | AveExpr | P.Value | adj.P.Val | miR_Sequence | SEQ ID NO: |
|---|---|---|---|---|---|---|---|
| 002352_hsa-miR-652_A | -0.57 | 0.67 | 25.85 | 0.0085 | 0.2495 | AAUGGCGCCACUAGGGUUGUG | 143 |
| 001274_hsa-miR-410_A | -0.47 | 0.72 | 25.47 | 0.0339 | 0.4367 | AAUAUAACACAGAUGGCCUGU | 144 |
| 000565_hsa-miR-376a_A | -0.42 | 0.75 | 22.95 | 0.0295 | 0.4367 | AUCAUAGAGGAAAAUCCACGU | 145 |
| 002422_hsa-miR-18a_A | -0.37 | 0.77 | 24.91 | 0.0101 | 0.2495 | UAAGGUGCAUCUAGUGCAGAUAG | 146 |
| 000436_hsa-miR-99b_A | -0.33 | 0.79 | 22.50 | 0.0088 | 0.2495 | CACCCGUAGAACCGACCUUGCG | 147 |
| 001187_mmu-miR-140_A | -0.27 | 0.83 | 23.16 | 0.0257 | 0.4367 | CAGUGGUUUUACCCUAUGGUAG | 148 |
| 000419_hsa-miR-30c_A | -0.27 | 0.83 | 18.26 | 0.0041 | 0.2388 | UGUAAACAUCCUACACUCUCAGC | 149 |
| 001138_mmu-miR-379_A | -0.26 | 0.83 | 27.64 | 0.0265 | 0.4367 | UGGUAGACUAUGGAACGUAGG | 150 |
| 000602_hsa-miR-30b_A | -0.22 | 0.86 | 18.16 | 0.0360 | 0.4367 | UGUAAACAUCCUACACUCAGCU | 151 |
| 001111_hsa-miR-511_A | -0.21 | 0.86 | 27.71 | 0.0302 | 0.4367 | GUGUCUUUUGCUCUGCAGUCA | 152 |
| 000395_hsa-miR-19a_A | 0.20 | 1.15 | 20.52 | 0.0409 | 0.4367 | UGUGCAAAUCUAUGCAAAACUGA | 153 |
| 002281_hsa-miR-193a-5p_A | 0.48 | 1.40 | 23.76 | 0.0092 | 0.2495 | UGGGUCUUUGCGGGCGAGAUGA | 154 |
| 002296_hsa-miR-885-5p_A | 0.49 | 1.41 | 20.38 | 0.0333 | 0.4367 | UCCAUUACACUACCCUGCCUCU | 155 |
| 002367_hsa-miR-193b_A | 0.53 | 1.44 | 20.83 | 0.0463 | 0.4367 | AACUGGCCCUCAAAGUCCCGCU | 156 |
| 002099_hsa-miR-224_A | 0.91 | 1.88 | 25.98 | 0.0001 | 0.0131 | CAAGUCACUAGUGGUUCCGUU | 157 |
| 000426_hsa-miR-34a_A | 1.04 | 2.06 | 23.56 | 0.0002 | 0.0131 | UGGCAGUGUCUUAGCUGGUUGU | 158 |

TABLE 13

| | | Linear | | | | | SEQ ID |
|---|---|---|---|---|---|---|---|
| ID | logFC | FC | AveExpr | P.Value | adj.P.Val | miR_Sequence | NO: |
| 000565_hsa-miR-376a_A | -0.55 | 0.68 | 22.95 | 0.0027 | 0.0769 | AUCAUAGAGGAAAAUCCACGU | 159 |
| 002352_hsa-miR-652_A | -0.52 | 0.70 | 25.85 | 0.0096 | 0.1472 | AAUGGCGCCACUAGGGUUGUG | 160 |
| 002122_hsa-miR-376c_A | -0.44 | 0.74 | 24.09 | 0.0359 | 0.2820 | AACAUAGAGGAAAUUCCACGU | 161 |
| 002422_hsa-miR-18a_A | -0.41 | 0.75 | 24.91 | 0.0022 | 0.0746 | UAAGGUGCAUCUAGUGCAGAUAG | 162 |
| 001274_hsa-miR-410_A | -0.41 | 0.75 | 25.47 | 0.0486 | 0.3219 | AAUAUAACACAGAUGGCCUGU | 163 |
| 002283_hsa-let-7d_A | -0.31 | 0.81 | 25.07 | 0.0215 | 0.2309 | AGAGGUAGUAGGUUGCAUAGUU | 164 |
| 000411_hsa-miR-28_A | -0.30 | 0.81 | 23.24 | 0.0111 | 0.1472 | AAGGAGCUCACAGUCUAUUGAG | 165 |
| 000602_hsa-miR-30b_A | -0.29 | 0.82 | 18.16 | 0.0031 | 0.0774 | UGUAAACAUCCUACACUCAGCU | 166 |
| 000419_hsa-miR-30c_A | -0.29 | 0.82 | 18.26 | 0.0008 | 0.0407 | UGUAAACAUCCUACACUCUCAGC | 167 |
| 001138_mmu-miR-379_A | -0.28 | 0.82 | 27.64 | 0.0105 | 0.1472 | UGGUAGACUAUGGAACGUAGG | 168 |
| 000539_hsa-miR-324-5p_A | -0.27 | 0.83 | 23.61 | 0.0415 | 0.3028 | CGCAUCCCCUAGGGCAUUGGUGU | 169 |
| 001187_mmu-miR-140_A | -0.26 | 0.83 | 23.16 | 0.0200 | 0.2303 | CAGUGGUUUUACCCUAUGGUAG | 170 |
| 000436_hsa-miR-99b_A | -0.26 | 0.83 | 22.50 | 0.0294 | 0.2640 | CACCCGUAGAACCGACCUUGCG | 171 |
| 001285_hsa-miR-487b_A | -0.13 | 0.91 | 27.84 | 0.0320 | 0.2640 | AAUCGUACAGGGUCAUCCACUU | 172 |
| 000395_hsa-miR-19a_A | 0.21 | 1.16 | 20.52 | 0.0240 | 0.2309 | UGUGCAAAUCUAUGCAAAACUGA | 173 |
| 002089_hsa-miR-505_A | 0.34 | 1.27 | 27.08 | 0.0229 | 0.2309 | CGUCAACACUUGCUGGUUUCCU | 174 |
| 000564_hsa-miR-375_A | 0.39 | 1.31 | 22.45 | 0.0420 | 0.3028 | UUUGUUCGUUCGGCUCGCGUGA | 175 |
| 002338_hsa-miR-483-5p_A | 0.48 | 1.39 | 21.10 | 0.0088 | 0.1472 | AAGACGGGAGGAAAGAAGGGAG | 176 |
| 000491_hsa-miR-192_A | 0.49 | 1.40 | 19.93 | 0.0176 | 0.2173 | CUGACCUAUGAAUUGACAGCC | 177 |
| 002245_hsa-miR-122_A | 0.49 | 1.41 | 19.47 | 0.0308 | 0.2640 | UGGAGUGUGACAAUGGUGUUUG | 178 |
| 002281_hsa-miR-193a- | 0.58 | 1.49 | 23.76 | 0.0009 | 0.0407 | UGGGUCUUUGCGGGCGAGAUGA | 179 |
| 5p_A | | | | | | | |
| 002296_hsa-miR-885-5p_A | 0.61 | 1.52 | 20.38 | 0.0053 | 0.1143 | UCCAUUACACUACCCUGCCUCU | 180 |
| 002367_hsa-miR-193b_A | 0.68 | 1.61 | 20.83 | 0.0064 | 0.1221 | AACUGGCCCUCAAAGUCCCGCU | 181 |
| 002099_hsa-miR-224_A | 1.07 | 2.11 | 25.98 | 2.52304E-06 | 0.0004 | CAAGUCACUAGUGGUUCCGUU | 182 |
| 000426_hsa-miR-34a_A | 1.17 | 2.25 | 23.56 | 7.22082E-06 | 0.0006 | UGGCAGUGUCUUAGCUGGUUGU | 183 |

TABLE 14

| ID | logFC | Linear FC | AveExpr | P.Value | adj.P.Val | miR_Sequence | SEQ ID NO: |
|---|---|---|---|---|---|---|---|
| 002299_hsa-miR-191_A | -0.51 | 0.70 | 18.60 | 0.0287 | 0.9182 | CAACGGAAUCCCAAAAGCAGCUG | 184 |
| 002302_hsa-miR-425-star_B | -0.46 | 0.73 | 27.14 | 0.0144 | 0.9182 | AUCGGGAAUGUCGUGUCCGCCC | 185 |
| 000411_hsa-miR-28_A | -0.38 | 0.77 | 23.24 | 0.0222 | 0.9182 | AAGGAGCUCACAGUCUAUUGAG | 186 |
| 000510_hsa-miR-206_B | 0.65 | 1.57 | 26.74 | 0.0485 | 0.9182 | UGGAAUGUAAGGAAGUGUGUGG | 187 |
| 002099_hsa-miR-224_A | 0.70 | 1.62 | 25.98 | 0.0226 | 0.9182 | CAAGUCACUAGUGGUUCCGUU | 188 |

TABLE 15

| ID | logFC | Linear FC | AveExpr | P. Value | adj. P. Val |
|---|---|---|---|---|---|
| 002099_hsa- | 0.91 | 1.88 | 25.98 | 0.0001 | 0.0131 |
| miR-224_A | | | | | |
| 000426_hsa- | 1.04 | 2.06 | 23.56 | 0.0002 | 0.0131 |
| miR-34a_A | | | | | |

TABLE 16

| ID | logFC | Linear FC | AveExpr | P. Value | adj. P. Val |
|---|---|---|---|---|---|
| 002099_hsa-miR-224_A | 1.59 | 3.01 | 25.98 | 8.27984E−06 | 0.001432413 |
| 000426_hsa-miR-34a_A | 1.56 | 2.96 | 23.56 | 0.0001 | 0.0108 |
| 001097_hsa-miR-146b_A | −0.71 | 0.61 | 22.17 | 0.0002 | 0.0141 |
| 000602_hsa-miR-30b_A | −0.53 | 0.69 | 18.16 | 0.0007 | 0.0292 |
| 000565_hsa-miR-376a_A | −0.96 | 0.51 | 22.95 | 0.0008 | 0.0292 |
| 002281_hsa-miR-193a-5p_A | 0.88 | 1.84 | 23.76 | 0.0013 | 0.0385 |
| 002367_hsa-miR-193b_A | 1.18 | 2.26 | 20.83 | 0.0029 | 0.0718 |
| 000411_hsa-miR-28_A | −0.54 | 0.69 | 23.24 | 0.0039 | 0.0824 |
| 002296_hsa-miR-885-5p_A | 0.97 | 1.96 | 20.38 | 0.0046 | 0.0824 |
| 002283_hsa-let-7d_A | −0.59 | 0.66 | 25.07 | 0.0053 | 0.0824 |
| 000475_hsa-miR-152_A | 0.46 | 1.37 | 22.71 | 0.0056 | 0.0824 |
| 002338_hsa-miR-483-5p_A | 0.79 | 1.73 | 21.10 | 0.0057 | 0.0824 |
| 000419_hsa-miR-30c_A | −0.37 | 0.77 | 18.26 | 0.0065 | 0.0863 |

TABLE 17

| ID | logFC | Linear FC | AveExpr | P. Value | adj. P. Val |
|---|---|---|---|---|---|
| 002099_hsa-miR-224_A | 1.77 | 3.42 | 25.98 | 3.58858E−08 | 6.20825E−06 |
| 000426_hsa-miR-34a_A | 1.51 | 2.85 | 23.56 | 3.16685E−05 | 0.002739328 |
| 000411_hsa-miR-28_A | −0.68 | 0.62 | 23.24 | 5.8745E−05 | 0.003387628 |
| 000419_hsa-miR-30c_A | −0.46 | 0.73 | 18.26 | 0.0002 | 0.0081 |
| 002281_hsa-miR-193a-5p_A | 0.83 | 1.78 | 23.76 | 0.0006 | 0.0215 |
| 000436_hsa-miR-99b_A | −0.55 | 0.68 | 22.50 | 0.0010 | 0.0297 |
| 000602_hsa-miR-30b_A | −0.44 | 0.74 | 18.16 | 0.0015 | 0.0362 |
| 002422_hsa-miR-18a_A | −0.55 | 0.68 | 24.91 | 0.0032 | 0.0689 |
| 002089_hsa-miR-505_A | 0.62 | 1.53 | 27.08 | 0.0037 | 0.0696 |
| 000564_hsa-miR-375_A | 0.79 | 1.73 | 22.45 | 0.0041 | 0.0696 |
| 002299_hsa-miR-191_A | −0.68 | 0.62 | 18.60 | 0.0044 | 0.0696 |
| 000521_hsa-miR-218_A | 0.73 | 1.66 | 26.35 | 0.0049 | 0.0703 |

TABLE 18

| | | | Geom mean | Geom mean | | |
|---|---|---|---|---|---|---|
| | | | of intensities | of intensities | | |
| | Parametric | | in Advanced | in No | Fold- | |
| | p-value | t-value | Fibrosis | Fibrosis | change | UniqueID |
| 1 | <1e−07 | −6.374 | 24.95 | 26.72 | 3.45 | 002099_hsa-miR-224_A |
| 2 | 0.0002638 | 3.813 | 23.68 | 23.00 | 0.63 | 000411_hsa-miR-28_A |
| 3 | 0.0002772 | −3.799 | 22.80 | 24.31 | 2.86 | 000426_hsa-miR-34a_A |
| 4 | 0.0004485 | 3.657 | 18.52 | 18.06 | 0.73 | 000419_hsa-miR-30c_A |
| 5 | 0.0008159 | −3.476 | 23.31 | 24.14 | 1.79 | 002281_hsa-miR-193a-5p_A |
| 6 | 0.0009571 | 3.426 | 25.20 | 24.64 | 0.68 | 002422_hsa-miR-18a_A |
| 7 | 0.0019948 | −3.193 | 26.71 | 27.33 | 1.54 | 002089_hsa-miR-505_A |
| 8 | 0.0021026 | 3.176 | 22.85 | 22.30 | 0.68 | 000436_hsa-miR-99b_A |
| 9 | 0.0023101 | 3.146 | 18.41 | 17.97 | 0.74 | 000602_hsa-miR-30b_A |
| 10 | 0.0057885 | −2.834 | 21.96 | 22.75 | 1.72 | 000564_hsa-miR-375_A |
| 11 | 0.0063076 | 2.803 | 27.96 | 27.49 | 0.72 | 002198_hsa-miR-125a-5p_A |
| 12 | 0.0065824 | −2.788 | 25.85 | 26.59 | 1.67 | 000521_hsa-miR-218_A |

TABLE 19

| | | | Diagonal | |
|---|---|---|---|---|
| | | Compound | Linear | Support |
| | | Covariate | Discriminant | Vector |
| | Genes | Predictor | Analysis | Machines |
| 1 | 000411_hsa-miR-28_A | 3.8134 | 1.3305 | 0.0596 |
| 2 | 000419_hsa-miR-30c_A | 3.6571 | 1.8735 | 0.6288 |
| 3 | 000426_hsa-miR-34a_A | −3.799 | −0.5809 | −0.3155 |
| 4 | 000436_hsa-miR-99b_A | 3.1759 | 1.1374 | 0.2633 |
| 5 | 000521_hsa-miR-218_A | −2.7881 | NA | −0.4358 |
| 6 | 000564_hsa-miR-375_A | −2.8335 | NA | −0.2309 |
| 7 | 000602_hsa-miR-30b_A | NA | NA | −0.2999 |
| 8 | 002089_hsa-miR-505_A | NA | NA | −0.0425 |
| 9 | 002099_hsa-miR-224_A | NA | NA | −0.5201 |
| 10 | 002198_hsa-miR-125a- | NA | NA | 0.6106 |
| | 5p_A | | | |
| 11 | 002281_hsa-miR-193a- | NA | NA | −0.0474 |
| | 5p_A | | | |
| 12 | 002422_hsa-miR-18a_A | NA | NA | 0.4429 |

TABLE 20

| Class | Sensitivity | Specificity | PPV | NPV |
|---|---|---|---|---|
| Advanced_Fibrosis | 0.727 | 0.783 | 0.552 | 0.887 |
| No_Fibrosis | 0.783 | 0.727 | 0.887 | 0.552 |

TABLE 21

| Class | Sensitivity | Specificity | PPV | NPV |
|---|---|---|---|---|
| Advanced_Fibrosis | 0.727 | 0.767 | 0.533 | 0.885 |
| No_Fibrosis | 0.767 | 0.727 | 0.885 | 0.533 |

TABLE 22

| Class | Sensitivity | Specificity | PPV | NPV |
|---|---|---|---|---|
| Advanced_Fibrosis | 0.5 | 0.917 | 0.688 | 0.833 |
| No_Fibrosis | 0.917 | 0.5 | 0.833 | 0.688 |

TABLE 23

| | | | | Geom mean | Geom mean | | |
|---|---|---|---|---|---|---|---|
| | | | | of intensities | of intensities | | |
| | | Parametric | | in Advanced | in No | Fold- | |
| | Pair | p-value | t-value | Fibrosis | Fibrosis | change | UniqueID |
| 1 | 1 | <1e−07 | −6.374 | 24.95 | 26.72 | 3.45 | 002099_hsa-miR-224_A |
| 2 | 1 | 0.0213223 | 2.347 | 19.10 | 18.42 | 0.63 | 002299_hsa-miR-191_A |

TABLE 24

| | | | Diagonal | |
|---|---|---|---|---|
| | | | Linear | |
| | | Compound | Discrim- | Support |
| | | Covariate | inant | Vector |
| | Genes | Predictor | Analysis | Machines |
| 1 | 002099_hsa-miR-224_A | −6.3741 | −1.3999 | −0.8806 |
| 2 | 002299_hsa-miR-191_A | 2.3471 | 0.4954 | 0.6605 |

TABLE 25

| Class | Sensitivity | Specificity | PPV | NPV |
|---|---|---|---|---|
| Advanced_Fibrosis | 0.727 | 0.833 | 0.615 | 0.893 |
| No_Fibrosis | 0.833 | 0.727 | 0.893 | 0.615 |

TABLE 26

| Class | Sensitivity | Specificity | PPV | NPV |
|---|---|---|---|---|
| Advanced_Fibrosis | 0.727 | 0.833 | 0.615 | 0.893 |
| No_Fibrosis | 0.833 | 0.727 | 0.893 | 0.615 |

TABLE 27

| Class | Sensitivity | Specificity | PPV | NPV |
|---|---|---|---|---|
| Advanced_Fibrosis | 0.545 | 0.983 | 0.923 | 0.855 |
| No_Fibrosis | 0.983 | 0.545 | 0.855 | 0.923 |

TABLE 28

| | | Linear | | | | | SEQ ID |
|---|---|---|---|---|---|---|---|
| ID | logFC | FC | AveExpr | P.Value | adj.P.Val | miR_Sequence | NO: |
| 000439_hsa-miR-103_A | -1.70 | 0.31 | 25.59 | 0.011177719 | 0.074374826 | AGCAGCAUUGUACAGGGCUAUGA | 189 |
| 002254_hsa-miR-151- | | | | | | | |
| 3p_B | -1.25 | 0.42 | 25.24 | 0.005889331 | 0.050452686 | CUAGACUGAAGCUCCUUGAGG | 190 |
| 001562_hsa-miR-629_B | -0.65 | 0.64 | 27.26 | 0.006707583 | 0.050452686 | GUUCUCCCAACGUAAGCCCAGC | 191 |
| 002098_hsa-miR-223- | | | | | | | |
| star_B | -0.65 | 0.64 | 24.55 | 0.005257721 | 0.050452686 | CGUGUAUUUGACAAGCUGAGUU | 192 |
| 002259_hsa-miR-340- | | | | | | | |
| star_B | -0.58 | 0.67 | 27.14 | 0.002405519 | 0.041615475 | UCCGUCUCAGUUACUUUAUAGC | 193 |
| 002295_hsa-miR-223_A | -0.56 | 0.68 | 13.31 | 0.003931111 | 0.048961953 | UGUCAGUUUGUCAAAUACCCCA | 194 |
| 002283_hsa-let-7d_A | -0.52 | 0.70 | 25.06 | 0.006688557 | 0.050452686 | AGAGGUAGUAGGUUGCAUAGUU | 195 |
| 000411_hsa-miR-28_A | -0.50 | 0.71 | 23.23 | 0.004484572 | 0.050452686 | AAGGAGCUCACAGUCUAUUGAG | 196 |
| 000528_hsa-miR-301_A | -0.50 | 0.71 | 23.87 | 0.006567714 | 0.050452686 | CAGUGCAAUAGUAUUGUCAAAGC | 197 |
| 000524_hsa-miR-221_A | -0.49 | 0.71 | 20.61 | 0.002079585 | 0.039974245 | AGCUACAUUGUCUGCUGGGUUUC | 198 |
| 000602_hsa-miR-30b_A | -0.41 | 0.75 | 18.16 | 0.005120507 | 0.050452686 | UGUAAACAUCCUACACUCAGCU | 199 |
| 001187_mmu-miR-140_A | -0.39 | 0.76 | 23.16 | 0.012944414 | 0.081679343 | CAGUGGUUUUACCCUAUGGUAG | 200 |
| 000419_hsa-miR-30c_A | -0.38 | 0.77 | 18.26 | 0.003164107 | 0.04561587 | UGUAAACAUCCUACACUCUCAGC | 201 |
| 001090_mmu-miR-93_A | -0.36 | 0.78 | 21.41 | 0.009455477 | 0.067474017 | CAAAGUGCUGUUCGUGCAGGUAG | 202 |
| 000442_hsa-miR-106b_A | -0.32 | 0.80 | 20.08 | 0.009750581 | 0.067474017 | UAAAGUGCUGACAGUGCAGAU | 203 |
| 000545_hsa-miR-331_A | -0.30 | 0.81 | 20.99 | 0.013691913 | 0.081679343 | GCCCCUGGGCCUAUCCUAGAA | 204 |
| 002169_hsa-miR-106a_A | -0.29 | 0.82 | 17.84 | 0.013325818 | 0.081679343 | AAAAGUGCUUACAGUGCAGGUAG | 205 |
| 000417_hsa-miR-30a- | 0.30 | 1.23 | 17.97 | 0.003962239 | 0.048961953 | UGUAAACAUCCUCGACUGGAAG | 206 |
| 5p_B | | | | | | | |
| 000475_hsa-miR-152_A | 0.57 | 1.49 | 22.71 | 9.33189E-05 | 0.002690695 | UCAGUGCAUGACAGAACUUGG | 207 |
| 002089_hsa-miR-505_A | 0.60 | 1.51 | 27.09 | 0.005489289 | 0.050452686 | CGUCAACACUUGCUGGUUUCCU | 208 |
| 002245_hsa-miR-122_A | 0.95 | 1.93 | 19.48 | 0.00307085 | 0.04561587 | UGGAGUGUGACAAUGGUGUUUG | 209 |
| 002281_hsa-miR-193a- | 0.95 | 1.93 | 23.77 | 0.000146216 | 0.003161924 | UGGGUCUUUGCGGGCGAGAUGA | 210 |
| 5p_A | | | | | | | |
| 002338_hsa-miR-483- | 0.97 | 1.95 | 21.11 | 0.000140126 | 0.003161924 | AAGACGGGAGGAAAGAAGGGAG | 211 |
| 5p_A | | | | | | | |
| 002296_hsa-miR-885- | 1.25 | 2.38 | 20.39 | 2.81231E-05 | 0.000989537 | UCCAUUACACUACCCUGCCUCU | 212 |
| 5p_A | | | | | | | |
| 000491_hsa-miR-192_A | 1.28 | 2.43 | 19.95 | 4.41872E-06 | 0.000254813 | CUGACCUAUGAAUUGACAGCC | 213 |
| 000426_hsa-miR-34a_A | 1.59 | 3.01 | 23.57 | 2.85993E-05 | 0.000989537 | UGGCAGUGUCUUAGCUGGUUGU | 214 |
| 002367_hsa-miR-193b_A | 1.60 | 3.03 | 20.84 | 3.33031E-06 | 0.000254813 | AACUGGCCCUCAAAGUCCCGCU | 215 |
| 002099_hsa-miR-224_A | 1.61 | 3.05 | 25.98 | 1.71712E-06 | 0.000254813 | CAAGUCACUAGUGGUUCCGUU | 216 |
| 002088_hsa-miR-636_A | 2.12 | 4.35 | 26.11 | 0.006278455 | 0.050452686 | UGUGCUUGCUCGUCCCGCCCGCA | 217 |

TABLE 29

| | | Linear | | | | | SEQ ID |
|---|---|---|---|---|---|---|---|
| ID | logFC | FC | AveExpr | P.Value | adj.P.Val | miR_Sequence | NO: |
| 000391_hsa-miR-16_A | -0.58 | 0.67 | 17.45 | 0.020995545 | 0.265286473 | UAGCAGCACGUAAAUAUUGGCG | 218 |
| 002259_hsa-miR-340- | -0.50 | 0.71 | 27.14 | 0.00189175 | 0.036363639 | UCCGUCUCAGUUACUUUAUAGC | 219 |
| star_B | | | | | | | |
| 002283_hsa-let-7d_A | -0.38 | 0.77 | 25.06 | 0.017850603 | 0.257346189 | AGAGGUAGUAGGUUGCAUAGUU | 220 |
| 000464_hsa-miR-142- | -0.38 | 0.77 | 19.89 | 0.00857382 | 0.134842801 | UGUAGUGUUUCCUACUUUAUGGA | 221 |
| 3p_A | | | | | | | |
| 002355_hsa-miR-532- | -0.32 | 0.80 | 26.53 | 0.041406261 | 0.421369593 | CCUCCCACACCCAAGGCUUGCA | 222 |
| 3p_A | | | | | | | |
| 000419_hsa-miR-30c_A | -0.21 | 0.87 | 18.26 | 0.04921032 | 0.42566927 | UGUAAACAUCCUACACUCUCAGC | 223 |
| 000417_hsa-miR-30a- | 0.20 | 1.15 | 17.97 | 0.024736166 | 0.285290452 | UGUAAACAUCCUCGACUGGAAG | 224 |
| 5p_B | | | | | | | |
| 002349_hsa-miR-574- | 0.24 | 1.18 | 22.43 | 0.035575101 | 0.384655784 | CACGCUCAUGCACACACCCACA | 225 |
| 3p_A | | | | | | | |
| 002863_HSA-MIR-1290_B | 0.28 | 1.21 | 27.64 | 0.046361977 | 0.422138003 | UGGAUUUUUGGAUCAGGGA | 226 |
| 000379_hsa-let-7c_A | 0.37 | 1.29 | 26.82 | 0.021468269 | 0.265286473 | UGAGGUAGUAGGUUGUAUGGUU | 227 |
| 000564_hsa-miR-375_A | 0.47 | 1.38 | 22.47 | 0.044341845 | 0.422138003 | UUUGUUCGUUCGGCUCGCGUGA | 228 |
| 000475_hsa-miR-152_A | 0.50 | 1.41 | 22.71 | 6.63009E-05 | 0.002867512 | UCAGUGCAUGACAGAACUUGG | 229 |
| 002281_hsa-miR-193a- | 0.65 | 1.57 | 23.77 | 0.001746309 | 0.036363639 | UGGGUCUUUGCGGGCGAGAUGA | 230 |
| 5p_A | | | | | | | |
| 002338_hsa-miR-483- | 0.67 | 1.60 | 21.11 | 0.001461828 | 0.036128035 | AAGACGGGAGGAAAGAAGGGAG | 231 |
| 5p_A | | | | | | | |
| 002245_hsa-miR-122_A | 0.81 | 1.75 | 19.48 | 0.002587109 | 0.044756977 | UGGAGUGUGACAAUGGUGUUUG | 232 |
| 000491_hsa-miR-192_A | 0.91 | 1.87 | 19.95 | 9.93997E-05 | 0.003439228 | CUGACCUAUGAAUUGACAGCC | 233 |
| 000426_hsa-miR-34a_A | 1.03 | 2.04 | 23.57 | 0.00111388 | 0.032116873 | UGGCAGUGUCUUAGCUGGUUGU | 234 |
| 002296_hsa-miR-885- | 1.07 | 2.10 | 20.39 | 2.18067E-05 | 0.001257521 | UCCAUUACACUACCCUGCCUCU | 235 |
| 5p_A | | | | | | | |
| 002099_hsa-miR-224_A | 1.33 | 2.51 | 25.98 | 2.49954E-06 | 0.000305678 | CAAGUCACUAGUGGUUCCGUU | 236 |
| 002367_hsa-miR-193b_A | 1.34 | 2.54 | 20.84 | 3.53385E-06 | 0.000305678 | AACUGGCCCUCAAAGUCCCGCU | 237 |

TABLE 30

| | | | | Geom mean | Geom mean | | |
|---|---|---|---|---|---|---|---|
| | | Parametric | | of intensities | of intensities | Fold- | |
| | Pair | p-value | t-value | in Score 0 | in Score 2 or 3 | change | UniqueID |
| 1 | 1 | 3.00E−07 | 5.743 | 21.31 | 19.71 | 3.03 | 002367_hsa-miR-193b_A |
| 2 | 1 | 0.0035766 | 3.026 | 27.18 | 25.06 | 4.35 | 002088_hsa-miR-636_A |
| 3 | 2 | 7.00E−06 | 4.899 | 26.46 | 24.86 | 3.05 | 002099_hsa-miR-224_A |
| 4 | 2 | 0.0016022 | −3.298 | 26.97 | 27.56 | 0.67 | 002259_hsa-miR-340-star_B |
| 5 | 3 | 1.62E−05 | 4.67 | 20.42 | 19.14 | 2.43 | 000491_hsa-miR-192_A |
| 6 | 3 | 0.0418779 | −2.077 | 26.42 | 26.80 | 0.77 | 002355_hsa-miR-532-3p_A |
| 7 | 4 | 2.26E−05 | 4.577 | 24.21 | 22.62 | 3.01 | 000426_hsa-miR-34a_A |
| 8 | 4 | 0.0400866 | −2.096 | 26.13 | 26.86 | 0.6 | 002278_hsa-miR-145_A |
| 9 | 5 | 6.05E−05 | 4.298 | 21.47 | 20.50 | 1.95 | 002338_hsa-miR-483-5p_A |
| 10 | 5 | 0.00025 | 3.886 | 22.87 | 22.29 | 1.49 | 000475_hsa-miR-152_A |
| 11 | 6 | 6.13E−05 | 4.295 | 20.75 | 19.50 | 2.38 | 002296_hsa-miR-885-5p_A |
| 12 | 6 | 0.0007561 | −3.54 | 24.15 | 24.80 | 0.64 | 002098_hsa-miR-223-star_B |
| 13 | 7 | 0.0004619 | −3.694 | 21.38 | 21.77 | 0.76 | 000390_hsa-miR-15b_A |
| 14 | 7 | 0.0067639 | −2.8 | 21.26 | 21.62 | 0.78 | 001090_mmu-miR-93_A |
| 15 | 8 | 0.0005252 | 3.655 | 24.13 | 23.18 | 1.93 | 002281_hsa-miR-193a-5p_A |
| 16 | 8 | 0.0014305 | −3.335 | 12.93 | 13.49 | 0.68 | 002295_hsa-miR-223_A |

TABLE 31

| | | | Diagonal | |
|---|---|---|---|---|
| | | | Linear | |
| | | Compound | Discrim- | Support |
| | | Covariate | inant | Vector |
| | Genes | Predictor | Analysis | Machines |
| 1 | 000390_hsa-miR-15b_A | −3.6944 | −2.3819 | 0.1952 |
| 2 | 000426_hsa-miR-34a_A | 4.5771 | 0.8174 | 0.5071 |
| 3 | 000475_hsa-miR-152_A | 3.8855 | 1.768 | −0.1672 |
| 4 | 000491_hsa-miR-192_A | 4.6698 | 1.0586 | 0.031 |
| 5 | 001090_mmu-miR-93_A | −2.8002 | −1.4499 | −0.9618 |
| 6 | 002088_hsa-miR-636_A | 3.0264 | 0.2654 | 0.6114 |
| 7 | 002099_hsa-miR-224_A | 4.8994 | 0.9262 | 0.6475 |
| 8 | 002278_hsa-miR-145_A | −2.096 | −0.3708 | −0.9328 |
| 9 | 002281_hsa-miR-193a-5p_A | 3.6545 | 0.8812 | 0.5507 |
| 10 | 002295_hsa-miR-223_A | −3.3349 | −1.2618 | 0.4059 |
| 11 | 002296_hsa-miR-885-5p_A | 4.2948 | 0.915 | −1.162 |
| 12 | 002338_hsa-miR-483-5p_A | 4.2984 | 1.2004 | 0.4014 |
| 13 | 002355_hsa-miR-532-3p_A | −2.0769 | −0.7214 | −0.417 |
| 14 | 002367_hsa-miR-193b_A | 5.7428 | 1.283 | 0.0694 |
| 15 | 002098_hsa-miR-223-star_B | −3.5402 | −1.2259 | −0.93 |
| 16 | 002259_hsa-miR-340-star_B | −3.2977 | −1.1842 | −0.5222 |

TABLE 32

| Class | Sensitivity | Specificity | PPV | NPV |
|---|---|---|---|---|
| score_0 | 0.788 | 0.7 | 0.743 | 0.75 |
| score_2_or_3 | 0.7 | 0.788 | 0.75 | 0.743 |

TABLE 33

| Class | Sensitivity | Specificity | PPV | NPV |
|---|---|---|---|---|
| score_0 | 0.818 | 0.667 | 0.73 | 0.769 |
| score_2_or_3 | 0.667 | 0.818 | 0.769 | 0.73 |

TABLE 34

| Class | Sensitivity | Specificity | PPV | NPV |
|---|---|---|---|---|
| score_0 | 0.727 | 0.767 | 0.774 | 0.719 |
| score_2_or_3 | 0.767 | 0.727 | 0.719 | 0.774 |

TABLE 35

| | | | | Geom mean | Geom mean | | |
|---|---|---|---|---|---|---|---|
| | | | | of | of | | |
| | | Parametric | | intensities | intensities | Fold- | |
| | Pair | p-value | t-value | in class 1 | in class 2 | change | UniqueID |
| 1 | 1 | 1.04E−05 | 4.615 | 26.19 | 24.86 | 2.51 | 002099_hsa-miR-224_A |
| 2 | 1 | 0.001787 | −3.2 | 27.06 | 27.56 | 0.71 | 002259_hsa-miR-340-star_B |
| 3 | 2 | 1.48E−05 | 4.528 | 21.06 | 19.71 | 2.54 | 002367_hsa-miR-193b_A |
| 4 | 2 | 0.0118753 | −2.557 | 19.81 | 20.19 | 0.77 | 000464_hsa-miR-142-3p_A |

TABLE 36

| | | | Diagonal | |
|---|---|---|---|---|
| | | | Linear | |
| | | Compound | Discrim- | Support |
| | | Covariate | inant | Vector |
| | Genes | Predictor | Analysis | Machines |
| 1 | 000464_hsa-miR-142-3p_A | −2.5573 | −0.7794 | −0.4317 |
| 2 | 002099_hsa-miR-224_A | 4.6154 | 0.7225 | 0.2112 |
| 3 | 002367_hsa-miR-193b_A | 4.5282 | 0.6882 | 0.3666 |
| 4 | 002259_hsa-miR-340-star_B | −3.1995 | −0.9154 | −0.3186 |

TABLE 37

| Class | Sensitivity | Specificity | PPV | NPV |
|---|---|---|---|---|
| score_1 | 0.753 | 0.667 | 0.865 | 0.488 |
| score_2_or_3 | 0.667 | 0.753 | 0.488 | 0.865 |

TABLE 38

| Class | Sensitivity | Specificity | PPV | NPV |
|---|---|---|---|---|
| score_1 | 0.753 | 0.633 | 0.853 | 0.475 |
| score_2_or_3 | 0.633 | 0.753 | 0.475 | 0.853 |

TABLE 39

| Class | Sensitivity | Specificity | PPV | NPV |
|---|---|---|---|---|
| score_1 | 0.859 | 0.233 | 0.76 | 0.368 |
| score_2_or_3 | 0.233 | 0.859 | 0.368 | 0.76 |

1. A method of characterizing the non-alcoholic fatty liver disease (NAFLD) state of a subject, comprising forming a biomarker panel having N micro-RNAs (miRNAs) selected from the differentially expressed miRNAs listed in at least one of Tables 1-4, 10-14, and 28-29, and detecting the level of each of the N miRNAs in the panel in a sample from the subject.

2. The method of claim 1, wherein N is from 1 to 20, from 1 to 5, from 6 to 10, from 11 to 15, or from 15 to 20.

3. A method of characterizing the NAFLD state in a subject, comprising detecting the level of at least one, at least two, at least three, at least four, at least five, at least six, at least seven, at least eight, at least nine, or at least ten or at least 15 miRNAs selected from the differentially increased and differentially decreased miRNAs listed in at least one of Tables 1-4, 10-14, and 28-29 in a sample from the subject;
wherein a level of at least one differentially increased miRNA that is higher than a control level of the respective miRNA and/or a level of at least one differentially decreased miRNA that is lower than a control level of the respective miRNA indicates the presence of NAFLD and/or the presence of a more advanced NAFLD state in the subject.

4. The method of any of claims 1-3, wherein characterizing the NAFLD state of the subject comprises characterizing the nonalcoholic steatohepatitis (NASH) state of the subject.

5. The method of claim 4, wherein the level of at least one, at least two, at least three, at least four, at least five, at least six, at least seven, at least eight, at least nine, or at least ten miRNAs selected from the differentially increased and differentially decreased miRNAs listed in at least one of Tables 1-4 is detected in the sample from the subject;
wherein a level of at least one differentially increased miRNA that is higher than a control level of the respective miRNA and/or a level of at least one differentially decreased miRNA that is lower than a control level of the respective miRNA indicates the presence of NASH and/or the presence of a more advanced stage of NASH in the subject.

6. The method of claim 5, wherein the NASH is stage 1, stage 2, stage 3 or stage 4 NASH.

7. The method of any of claims 1-3, wherein characterizing the NAFLD state of the subject comprises characterizing the occurrence of liver fibrosis in the subject.

8. The method of claim 7, wherein the level of at least one, at least two, at least three, at least four, at least five, at least six, at least seven, at least eight, at least nine, or at least ten miRNAs selected from the differentially increased and differentially decreased miRNAs listed in at least one of Tables 10-14 is detected in the sample from the subject;
wherein a level of at least one differentially increased miRNA that is higher than a control level of the respective miRNA and/or a level of at least one differentially decreased miRNA that is lower than a control level of the respective miRNA indicates the presence of liver fibrosis and/or the presence of more advanced liver fibrosis in the subject.

9. The method of any of claims 1-3, wherein characterizing the NAFLD state of the subject comprises characterizing the occurrence of hepatocellular ballooning in the subject.

10. The method of claim 9, wherein detecting the level of at least one, at least two, at least three, at least four, at least five, at least six, at least seven, at least eight, at least nine, or at least ten miRNAs selected from the differentially increased and differentially decreased miRNAs listed in at least one of Tables 28 and 29 is detected in the sample from the subject;
wherein a level of at least one differentially increased miRNA that is higher than a control level of the respective miRNA and/or a level of at least one differentially decreased miRNA that is lower than a control level of the respective miRNA indicates the presence of hepatocellular ballooning and/or the presence of more advanced hepatocellular ballooning in the subject.

11. A method of determining whether a subject has NASH, comprising
providing a sample from a subject suspected of NASH;
forming a biomarker panel having N micro-RNAs miRNAs selected from the differentially increased and differentially decreased miRNAs listed in at least one of Tables 1-4; and
detecting the level of each of the N miRNAs in the panel in the sample from the subject.

12. The method of claim 11, wherein N is from 1 to 20, from 1 to 5, from 6 to 10, from 11 to 15, or from 15 to 20.

13. A method of determining whether a subject has NASH, comprising providing a sample from a subject suspected of NASH and detecting the level of at least one, at least two, at least three, at least four, at least five, at least six, at least seven, at least eight, at least nine, or at least ten miRNAs selected from the differentially increased and differentially decreased miRNAs listed in at least one of Tables 1-4 in the sample from the subject;
wherein a level of at least one differentially increased miRNA that is higher than a control level of the respective miRNA and/or a level of at least one differentially decreased miRNA that is lower than a control level of the respective miRNA indicates that the subject has NASH.

14. The method of claim 13, comprising detecting the level of at least one pair of miRNAs selected from pairs 1-10 listed in Table 5 in the sample from the subject.

15. The method of claim 13, wherein the sample is from a subject diagnosed with mild, moderate, or severe NAFLD.

16. The method of claim 13, wherein the subject is not previously diagnosed with NASH.

17. The method of claim 13, wherein the NASH is stage 1, 2, 3, or 4 NASH.

18. The method of any one of claim 13, wherein the subject is previously diagnosed with NAFLD.

19. The method of claim 18, wherein the sample is from a subject diagnosed with mild, moderate, or severe NAFLD.

20. The method of claim 18, wherein the subject has presented with at least one clinical symptom of NASH.

21. A method of monitoring NASH therapy in a subject, comprising
providing a sample from a subject undergoing treatment for NASH;
forming a biomarker panel having N micro-RNAs miRNAs selected from the differentially increased and differentially decreased miRNAs listed in at least one of Tables 1-4; and
detecting the level of each of the N miRNAs in the panel in the sample from the subject.

22. The method of claim 21, wherein N is from 1 to 20, from 1 to 5, from 6 to 10, from 11 to 15, or from 15 to 20.

23. A method of monitoring NASH therapy in a subject, comprising providing a sample from a subject undergoing treatment for NASH and detecting the level of at least one, at least two, at least three, at least four, at least five, at least six, at least seven, at least eight, at least nine, or at least ten miRNAs selected from the differentially increased and differentially decreased miRNAs listed in at least one of Tables 1-4 in the sample from the subject;
wherein a level of at least one differentially increased miRNA that is higher than a control level of the respective miRNA and/or a level of at least one differentially decreased miRNA that is lower than a control level of the respective miRNA indicates that the NASH is increasing in severity; and
wherein the absence of a level of at least one differentially increased miRNA that is higher than a control level of the respective miRNA and/or a level of at least one differentially decreased miRNA that is lower than a control level of the respective miRNA indicates that the NASH is not increasing in severity.

24. The method of claim 23, comprising detecting the level of at least one pair of miRNAs selected from pairs 1-10 listed in Table 5 in the sample from the subject.

25. The method of claim 23, wherein the NASH is stage 1, 2, 3, or 4 NASH.

26. A method of characterizing the risk that a subject with NAFLD will develop NASH, comprising providing a sample from a subject suspected with NAFLD and detecting the level of at least one, at least two, at least three, at least four, at least five, at least six, at least seven, at least eight, at least nine, or at least ten miRNAs selected from the differentially increased and differentially decreased miRNAs listed in at least one of Tables 1-4 in the sample from the subject;
wherein a level of at least one differentially increased miRNA that is higher than a control level of the respective miRNA and/or a level of at least one differentially decreased miRNA that is lower than a control level of the respective miRNA indicates an increased risk that the subject will develop NASH; and/or
wherein the absence of a level of at least one differentially increased miRNA that is higher than a control level of the respective miRNA and/or a level of at least one differentially decreased miRNA that is lower than a control level of the respective miRNA indicates a decreased risk that the subject will develop NASH.

27. The method of claim 26, comprising detecting the level of at least one pair of miRNAs selected from pairs 1-10 listed in Table 5 in the sample from the subject.

28. The method of claim 26, wherein the sample is from a subject diagnosed with mild, moderate, or severe NAFLD.

29. A method of determining whether a subject has liver fibrosis, comprising
providing a sample from a subject suspected of liver fibrosis;
forming a biomarker panel having N miRNAs selected from the differentially increased and differentially decreased miRNAs listed in at least one of Tables 10-14; and
detecting the level of each of the N miRNAs in the panel in the sample from the subject.

30. The method of claim 29, wherein N is from 1 to 20, from 1 to 5, from 6 to 10, from 11 to 15, or from 15 to 20.

31. A method of determining whether a subject has liver fibrosis, comprising providing a sample from a subject suspected of liver fibrosis and detecting the level of at least one, at least two, at least three, at least four, at least five, at least six, at least seven, at least eight, at least nine, or at least ten miRNAs selected from the differentially increased and differentially decreased miRNAs listed in at least one of Tables 10-14;
wherein a level of at least one differentially increased miRNA that is higher than a control level of the respective miRNA and/or a level of at least one differentially decreased miRNA that is lower than a control level of the respective miRNA indicates the presence of liver fibrosis.

32. The method of claim 31, comprising detecting the level of at least one miRNA selected from the differentially increased and differentially decreased miRNAs listed in at least one of Tables 15-17.

33. The method of claim 32, wherein the at least one miRNA is miR-224.

34. The method of claim 31, comprising detecting the level of at least one miRNA selected from the differentially increased and differentially decreased miRNAs listed in Table 18.

35. The method of claim 31, comprising detecting the level of miR-224 and/or miR-191.

36. The method of claim 31, wherein the liver fibrosis is stage 1, 2, 3, or 4 liver fibrosis.

37. The method of claim 31, wherein the sample is from a subject diagnosed with mild, moderate, or severe NAFLD.

38. The method of claim 31, wherein the sample is from a subject diagnosed with NASH.

39. The method of claim 39, wherein the NASH is stage 1, 2, 3, or 4 NASH.

40. A method of determining whether a subject has hepatocellular ballooning, comprising
providing a sample from a subject suspected of hepatocellular ballooning;
forming a biomarker panel having N miRNAs selected from the differentially increased and differentially decreased miRNAs listed in at least one of Tables 28 and 29; and
detecting the level of each of the N miRNAs in the panel in the sample from the subject.

41. The method of claim 40, wherein N is from 1 to 20, from 1 to 5, from 6 to 10, from 11 to 15, or from 15 to 20.

42. A method of determining whether a subject has hepatocellular ballooning, comprising providing a sample from a subject suspected of hepatocellular ballooning and detecting the level of at least one, at least two, at least three, at least four, at least five, at least six, at least seven, at least eight, at least nine, or at least ten miRNAs selected from the differentially increased and differentially decreased miRNAs listed in at least one of Tables 28 and 29 in the sample from the subject;
wherein a level of at least one differentially increased miRNA that is higher than a control level of the respective miRNA and/or a level of at least one differentially decreased miRNA that is lower than a control level of the respective miRNA indicates the presence of hepatocellular ballooning.

43. The method of claim 42, comprising detecting the level of at least one pair of miRNAs selected from the pairs listed in Table 30 in the sample from the subject.

44. The method of claim 42, comprising detecting the level of at least one pair of miRNAs selected from the pairs listed in Table 35 in the sample from the subject.

45. The method of claim 42, wherein the sample is from a subject diagnosed with mild, moderate, or severe NAFLD.

46. The method of claim 42, wherein the sample is from a subject diagnosed with NASH.

47. The method of claim 46, wherein the NASH is stage 1, 2, 3, or 4 NASH.

48. The method of any one of the preceding claims, wherein the detecting comprises RT-PCR.

49. The method of claim 48, wherein the detecting comprises quantitative RT-PCR.

50. The method of any one of the preceding claims, wherein the sample is a bodily fluid.

51. The method of claim 50, wherein the sample is selected from blood, a blood component, urine, sputum, saliva, and mucus.

52. The method of claim 51, wherein the sample is serum.

53. The method of any preceding claim, wherein the method comprises characterizing the NAFLD or NASH state of the subject for the purpose of determining a medical insurance premium or a life insurance premium.

54. The method of claim 53, further comprising determining a medical insurance premium or a life insurance premium for the subject.

55. A composition comprising:
RNAs of a sample from a subject or cDNAs reverse transcribed from the RNAs of a sample from a subject; and
a set of polynucleotides for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 1-4, 10-14, and 28-29.

56. The composition of claim 55, wherein the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 1-4.

57. The composition of claim 55, wherein the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 10-14.

58. The composition of claim 55, wherein the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 28 and 29.

59. The composition of any one of claims 55 to 58, wherein each polynucleotide independently comprises from 8 to 100, from 8 to 75, from 8 to 50, from 8 to 40, from 8 to 30, from 12 to 100, from 12 to 75, from 12 to 50, from 12 to 40, or from 12 to 30 nucleotides.

60. The composition of any one of claims 55 to 58, wherein the sample is a bodily fluid.

61. The composition of claim 63, wherein the sample is selected from blood, a blood component, urine, sputum, saliva, and mucus.

62. The composition of claim 64, wherein the sample is serum.

63. A kit comprising
a set of polynucleotides for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 1-4, 10-14, and 28-29.

64. The kit of claim 63, wherein the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 1-4.

65. The kit of claim 63, wherein the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 10-14.

66. The kit of claim 63, wherein the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 28 and 29.

67. The kit of any one of claims 63 to 66, wherein each polynucleotide independently comprises from 8 to 100, from 8 to 75, from 8 to 50, from 8 to 40, from 8 to 30, from 12 to 100, from 12 to 75, from 12 to 50, from 12 to 40, or from 12 to 30 nucleotides.

68. The kit of any one of claims 63 to 67, wherein the polynucleotides are packages for use in a multiplex assay.

69. The kit of any one of claims 63 to 67, wherein the polynucleotides are packages for use in a non-multiplex assay.

70. A system comprising:
a set of polynucleotides for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 1-4, 10-14, and 28-29; and
RNAs of a sample from a subject or cDNAs reverse transcribed from the RNAs of a sample from a subject.

71. The system of claim 70, wherein the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 1-4.

72. The system of claim 70, wherein the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 10-14.

74. The system of claim 70, wherein the set of polynucleotides is for detecting at least one, at least two, at least three, at least four, at least five, at least six, at least seven, at least eight, at least nine, or ten RNAs selected from the group consisting of miRNAs selected from the differentially increased and differentially decreased miRNAs listed in at least one of Tables 28 and 29.

75. The system of any one of claims 70 to 74, wherein each polynucleotide independently comprises from 8 to 100, from 8 to 75, from 8 to 50, from 8 to 40, from 8 to 30, from 12 to 100, from 12 to 75, from 12 to 50, from 12 to 40, or from 12 to 30 nucleotides

76. The system of any one of claims 70 to 75, wherein the sample is a bodily fluid.

77. The system of claim 76, wherein the sample is selected from blood, a blood component, urine, sputum, saliva, and mucus.

78. The system of claim 77, wherein the sample is serum.

79. The system of any one of claims 70-75, wherein the RNAs of a sample from a subject or cDNAs reverse transcribed from the RNAs of a sample from a subject are in a container, and wherein the set of polynucleotides is packaged separately from the container.


SEQUENCE LISTING (237 sequences)

SEQ ID NO: 1 - RNA, 23 nt, Homo sapiens
agcagcauuguacagggcuauga

SEQ ID NO: 2 - RNA, 23 nt, Homo sapiens
ucccuguccuccaggagcucacg

SEQ ID NO: 3 - RNA, 22 nt, Mus musculus
auauaauacaaccugcuaagug

SEQ ID NO: 4 - RNA, 23 nt, Homo sapiens
guccaguuuucccaggaaucccu

SEQ ID NO: 5 - RNA, 22 nt, Homo sapiens
acuccagccccacagccucagc

SEQ ID NO: 6 - RNA, 22 nt, Homo sapiens
guucucccaacguaagcccagc

SEQ ID NO: 7 - RNA, 23 nt, Homo sapiens
caacggaaucccaaaagcagcug

SEQ ID NO: 8 - RNA, 21 nt, Homo sapiens
aucauagaggaaaauccacgu

SEQ ID NO: 9 - RNA, 22 nt, Homo sapiens
aaggagcucacagucuauugag

SEQ ID NO: 10 - RNA, 23 nt, Homo sapiens
cagugcaauaguauugucaaagc

SEQ ID NO: 11 - RNA, 22 nt, Homo sapiens
agagguaguagguugcauaguu

SEQ ID NO: 12 - RNA, 23 nt, Homo sapiens
uguaaacauccuacacucucagc

SEQ ID NO: 13 - RNA, 22 nt, Homo sapiens
uguaaacauccuacacucagcu

SEQ ID NO: 14 - RNA, 23 nt, Homo sapiens
uaaggugcaucuagugcagauag

SEQ ID NO: 15 - RNA, 22 nt, Homo sapiens
ggagaaauuauccuuggugugu

SEQ ID NO: 16 - RNA, 23 nt, Homo sapiens
agcuacauugucugcuggguuuc

SEQ ID NO: 17 - RNA, 22 nt, Homo sapiens
uccgucucaguuacuuuauagc

SEQ ID NO: 18 - RNA, 22 nt, Homo sapiens
cacccguagaaccgaccuugcg

SEQ ID NO: 19 - RNA, 21 nt, Homo sapiens
gccccugggccuauccuagaa

SEQ ID NO: 20 - RNA, 24 nt, Homo sapiens
ucccugagacccuuuaaccuguga

SEQ ID NO: 21 - RNA, 22 nt, Homo sapiens
ucguaccgugaguaauaaugcg

SEQ ID NO: 22 - RNA, 22 nt, Homo sapiens
cuggcccucucugcccuuccgu

SEQ ID NO: 23 - RNA, 22 nt, Homo sapiens
aaucguacagggucauccacuu

SEQ ID NO: 24 - RNA, 22 nt, Homo sapiens
uguaaacauccccgacuggaag

SEQ ID NO: 25 - RNA, 22 nt, Homo sapiens
uguaaacauccucgacuggaag

SEQ ID NO: 26 - RNA, 21 nt, Homo sapiens
ucagugcaugacagaacuugg

SEQ ID NO: 27 - RNA, 22 nt, Homo sapiens
uacccauugcauaucggaguug

SEQ ID NO: 28 - RNA, 21 nt, Homo sapiens
cugaccuaugaauugacagcc

SEQ ID NO: 29 - RNA, 22 nt, Homo sapiens
aacuggcccucaaagucccgcu

SEQ ID NO: 30 - RNA, 22 nt, Homo sapiens
cgucaacacuugcugguuuccu

SEQ ID NO: 31 - RNA, 22 nt, Homo sapiens
ugggucuuugcgggcgagauga

SEQ ID NO: 32 - RNA, 21 nt, Homo sapiens
caagucacuagugguuccguu

SEQ ID NO: 33 - RNA, 22 nt, Homo sapiens
uggcagugucuuagcugguugu

SEQ ID NO: 34 - RNA, 23 nt, Homo sapiens
agcagcauuguacagggcuauga

SEQ ID NO: 35 - RNA, 23 nt, Homo sapiens
guccaguuuucccaggaaucccu

SEQ ID NO: 36 - RNA, 21 nt, Homo sapiens
aauggcgccacuaggguugug

SEQ ID NO: 37 - RNA, 22 nt, Homo sapiens
aaggagcucacagucuauugag

SEQ ID NO: 38 - RNA, 23 nt, Homo sapiens
gcaaagcacacggccugcagaga

SEQ ID NO: 39 - RNA, 23 nt, Homo sapiens
caacggaaucccaaaagcagcug

SEQ ID NO: 40 - RNA, 23 nt, Homo sapiens
cagugcaauaguauugucaaagc

SEQ ID NO: 41 - RNA, 22 nt, Homo sapiens
uccgucucaguuacuuuauagc

SEQ ID NO: 42 - RNA, 22 nt, Homo sapiens
ugucaguuugucaaauacccca

SEQ ID NO: 43 - RNA, 22 nt, Homo sapiens
caaagaauucuccuuuugggcu

SEQ ID NO: 44 - RNA, 23 nt, Homo sapiens
uguaaacauccuacacucucagc

SEQ ID NO: 45 - RNA, 23 nt, Homo sapiens
agcuacauugucugcuggguuuc

SEQ ID NO: 46 - RNA, 22 nt, Homo sapiens
uguaaacauccuacacucagcu

SEQ ID NO: 47 - RNA, 21 nt, Homo sapiens
ucgaggagcucacagucuagu

SEQ ID NO: 48 - RNA, 21 nt, Homo sapiens
gccccugggccuauccuagaa

SEQ ID NO: 49 - RNA, 22 nt, Homo sapiens
cuggcccucucugcccuuccgu

SEQ ID NO: 50 - RNA, 22 nt, Homo sapiens
accacugaccguugacuguacc

SEQ ID NO: 51 - RNA, 22 nt, Homo sapiens
aaaagcuggguugagagggcga

SEQ ID NO: 52 - RNA, 22 nt, Homo sapiens
uacccauugcauaucggaguug

SEQ ID NO: 53 - RNA, 22 nt, Homo sapiens
cgucaacacuugcugguuuccu

SEQ ID NO: 54 - RNA, 22 nt, Homo sapiens
aaaagcuggguugagagggcaa

SEQ ID NO: 55 - RNA, 22 nt, Homo sapiens
uucaacggguauuuauugagca

SEQ ID NO: 56 - RNA, 21 nt, Homo sapiens
cugaccuaugaauugacagcc

SEQ ID NO: 57 - RNA, 22 nt, Homo sapiens
uggcagugucuuagcugguugu

SEQ ID NO: 58 - RNA, 22 nt, Homo sapiens
guucucccaacguaagcccagc

SEQ ID NO: 59 - RNA, 22 nt, Homo sapiens
cacccguagaaccgaccuugcg

SEQ ID NO: 60 - RNA, 22 nt, Homo sapiens
agagguaguagguugcauaguu

SEQ ID NO: 61 - RNA, 23 nt, Homo sapiens
uguaaacauccuacacucucagc

SEQ ID NO: 62 - RNA, 22 nt, Homo sapiens
uguaaacauccuacacucagcu

SEQ ID NO: 63 - RNA, 22 nt, Homo sapiens
ggagaaauuauccuuggugugu

SEQ ID NO: 64 - RNA, 21 nt, Homo sapiens
gccccugggccuauccuagaa

SEQ ID NO: 65 - RNA, 22 nt, Homo sapiens
ucuacagugcacgugucuccag

SEQ ID NO: 66 - RNA, 22 nt, Homo sapiens
aaucguacagggucauccacuu

SEQ ID NO: 67 - RNA, 22 nt, Homo sapiens
uguaaacauccccgacuggaag

SEQ ID NO: 68 - RNA, 22 nt, Homo sapiens
uguaaacauccucgacuggaag

SEQ ID NO: 69 - RNA, 22 nt, Homo sapiens
gagcuuauucauaaaagugcag

SEQ ID NO: 70 - RNA, 22 nt, Homo sapiens
uggagugugacaaugguguuug

SEQ ID NO: 71 - RNA, 22 nt, Homo sapiens
ugggucuuugcgggcgagauga

SEQ ID NO: 72 - RNA, 22 nt, Homo sapiens
cgucaacacuugcugguuuccu

SEQ ID NO: 73 - RNA, 21 nt, Homo sapiens
caagucacuagugguuccguu

SEQ ID NO: 74 - RNA, 22 nt, Homo sapiens
uggcagugucuuagcugguugu

SEQ ID NO: 75 - RNA, 21 nt, Homo sapiens
aauggcgccacuaggguugug

SEQ ID NO: 76 - RNA, 23 nt, Homo sapiens
uagcaccauuugaaaucaguguu

SEQ ID NO: 77 - RNA, 22 nt, Homo sapiens
caaagaauucuccuuuugggcu

SEQ ID NO: 78 - RNA, 21 nt, Homo sapiens
ucgaggagcucacagucuagu

SEQ ID NO: 79 - RNA, 22 nt, Homo sapiens
accacugaccguugacuguacc

SEQ ID NO: 80 - RNA, 22 nt, Homo sapiens
cacccguagaaccgaccuugcg

SEQ ID NO: 81 - RNA, 22 nt, Homo sapiens
aaaagcuggguugagagggcga

SEQ ID NO: 82 - RNA, 22 nt, Homo sapiens
aaaagcuggguugagagggcaa

SEQ ID NO: 83 - RNA, 22 nt, Homo sapiens
uucaacggguauuuauugagca

SEQ ID NO: 84 - RNA, 21 nt, Homo sapiens
acuggacuuggagucagaagg

SEQ ID NO: 85 - RNA, 23 nt, Homo sapiens
agcagcauuguacagggcuauga

SEQ ID NO: 86 - RNA, 23 nt, Homo sapiens
ucccuguccuccaggagcucacg

SEQ ID NO: 87 - RNA, 22 nt, Homo sapiens
aaggagcucacagucuauugag

SEQ ID NO: 88 - RNA, 23 nt, Homo sapiens
caacggaaucccaaaagcagcug

SEQ ID NO: 89 - RNA, 21 nt, Homo sapiens
aacauagaggaaauuccacgu

SEQ ID NO: 90 - RNA, 21 nt, Homo sapiens
aucauagaggaaaauccacgu

SEQ ID NO: 91 - RNA, 23 nt, Homo sapiens
uaaggugcaucuagugcagauag

SEQ ID NO: 92 - RNA, 22 nt, Homo sapiens
cacccguagaaccgaccuugcg

SEQ ID NO: 93 - RNA, 24 nt, Homo sapiens
ucccugagacccuuuaaccuguga

SEQ ID NO: 94 - RNA, 23 nt, Homo sapiens
uguaaacauccuacacucucagc

SEQ ID NO: 95 - RNA, 22 nt, Homo sapiens
uguaaacauccuacacucagcu

SEQ ID NO: 96 - RNA, 22 nt, Homo sapiens
agagguaguagguugcauaguu

SEQ ID NO: 97 - RNA, 22 nt, Homo sapiens
uccgucucaguuacuuuauagc

SEQ ID NO: 98 - RNA, 21 nt, Homo sapiens
gccccugggccuauccuagaa

SEQ ID NO: 99 - RNA, 22 nt, Homo sapiens
cuggcccucucugcccuuccgu

SEQ ID NO: 100 - RNA, 22 nt, Homo sapiens
uguaaacauccucgacuggaag

SEQ ID NO: 101 - RNA, 22 nt, Homo sapiens
uucaacggguauuuauugagca

SEQ ID NO: 102 - RNA, 22 nt, Homo sapiens
cgucaacacuugcugguuuccu

SEQ ID NO: 103 - RNA, 22 nt, Homo sapiens
ucccugagacccuaacuuguga

SEQ ID NO: 104 - RNA, 21 nt, Homo sapiens
cugaccuaugaauugacagcc

SEQ ID NO: 105 - RNA, 22 nt, Homo sapiens
uccauuacacuacccugccucu

SEQ ID NO: 106 - RNA, 21 nt, Homo sapiens
uugugcuugaucuaaccaugu

SEQ ID NO: 107 - RNA, 22 nt, Homo sapiens
aacuggcccucaaagucccgcu

SEQ ID NO: 108 - RNA, 22 nt, Homo sapiens
uuuguucguucggcucgcguga

SEQ ID NO: 109 - RNA, 22 nt, Homo sapiens
ugggucuuugcgggcgagauga

SEQ ID NO: 110 - RNA, 22 nt, Homo sapiens
uggcagugucuuagcugguugu

SEQ ID NO: 111 - RNA, 21 nt, Homo sapiens
caagucacuagugguuccguu

SEQ ID NO: 112 - RNA, 22 nt, Homo sapiens
uggucuaggauuguuggaggag

SEQ ID NO: 113 - RNA, 23 nt, Homo sapiens
ucccuguccuccaggagcucacg

SEQ ID NO: 114 - RNA, 23 nt, Homo sapiens
uagugcaauauugcuuauagggu

SEQ ID NO: 115 - RNA, 21 nt, Homo sapiens
aucauagaggaaaauccacgu

SEQ ID NO: 116 - RNA, 22 nt, Homo sapiens
ugagaacugaauuccauaggcu

SEQ ID NO: 117 - RNA, 22 nt, Homo sapiens
agagguaguagguugcauaguu

SEQ ID NO: 118 - RNA, 23 nt, Homo sapiens
uaaggugcaucuagugcagauag

SEQ ID NO: 119 - RNA, 22 nt, Homo sapiens
aaggagcucacagucuauugag

SEQ ID NO: 120 - RNA, 22 nt, Homo sapiens
uguaaacauccuacacucagcu

SEQ ID NO: 121 - RNA, 22 nt, Homo sapiens
ccucccacacccaaggcuugca

SEQ ID NO: 122 - RNA, 22 nt, Homo sapiens
ugcggggcuagggcuaacagca

SEQ ID NO: 123 - RNA, 23 nt, Homo sapiens
uguaaacauccuacacucucagc

SEQ ID NO: 124 - RNA, 23 nt, Homo sapiens
agcuacauugucugcuggguuuc

SEQ ID NO: 125 - RNA, 22 nt, Homo sapiens
ugagaacugaauuccauggguu

SEQ ID NO: 126 - RNA, 21 nt, Mus musculus
ugguagacuauggaacguagg

SEQ ID NO: 127 - RNA, 22 nt, Homo sapiens
ucguaccgugaguaauaaugcg

SEQ ID NO: 128 - RNA, 22 nt, Homo sapiens
aaaagcuggguugagagggcga

SEQ ID NO: 129 - RNA, 21 nt, Homo sapiens
ucagugcaugacagaacuugg

SEQ ID NO: 130 - RNA, 22 nt, Homo sapiens
ugugucacucgaugaccacugu

SEQ ID NO: 131 - RNA, 22 nt, Homo sapiens
gacuauagaacuuucccccuca

SEQ ID NO: 132 - RNA, 22 nt, Homo sapiens
uggagugugacaaugguguuug

SEQ ID NO: 133 - RNA, 22 nt, Homo sapiens
uaaugccccuaaaaauccuuau

SEQ ID NO: 134 - RNA, 22 nt, Homo sapiens
aagacgggaggaaagaagggag

SEQ ID NO: 135 - RNA, 21 nt, Homo sapiens
cugaccuaugaauugacagcc

SEQ ID NO: 136 - RNA, 22 nt, Homo sapiens
ugggucuuugcgggcgagauga

SEQ ID NO: 137 - RNA, 22 nt, Homo sapiens
uccauuacacuacccugccucu

SEQ ID NO: 138 - RNA, 21 nt, Homo sapiens
uaacagucuccagucacggcc

SEQ ID NO: 139 - RNA, 22 nt, Homo sapiens
aacuggcccucaaagucccgcu

SEQ ID NO: 140 - RNA, 23 nt, Homo sapiens
ucucacacagaaaucgcacccgu

SEQ ID NO: 141 - RNA, 22 nt, Homo sapiens
uggcagugucuuagcugguugu

SEQ ID NO: 142 - RNA, 21 nt, Homo sapiens
caagucacuagugguuccguu

SEQ ID NO: 143 - RNA, 21 nt, Homo sapiens
aauggcgccacuaggguugug

SEQ ID NO: 144 - RNA, 21 nt, Homo sapiens
aauauaacacagauggccugu

SEQ ID NO: 145 - RNA, 21 nt, Homo sapiens
aucauagaggaaaauccacgu

SEQ ID NO: 146 - RNA, 23 nt, Homo sapiens
uaaggugcaucuagugcagauag

SEQ ID NO: 147 - RNA, 22 nt, Homo sapiens
cacccguagaaccgaccuugcg

SEQ ID NO: 148 - RNA, 22 nt, Mus musculus
cagugguuuuacccuaugguag

SEQ ID NO: 149 - RNA, 23 nt, Homo sapiens
uguaaacauccuacacucucagc

SEQ ID NO: 150 - RNA, 21 nt, Mus musculus
ugguagacuauggaacguagg

SEQ ID NO: 151 - RNA, 22 nt, Homo sapiens
uguaaacauccuacacucagcu

SEQ ID NO: 152 - RNA, 21 nt, Homo sapiens
gugucuuuugcucugcaguca

SEQ ID NO: 153 - RNA, 23 nt, Homo sapiens
ugugcaaaucuaugcaaaacuga

SEQ ID NO: 154 - RNA, 22 nt, Homo sapiens
ugggucuuugcgggcgagauga

SEQ ID NO: 155 - RNA, 22 nt, Homo sapiens
uccauuacacuacccugccucu

SEQ ID NO: 156 - RNA, 22 nt, Homo sapiens
aacuggcccucaaagucccgcu

SEQ ID NO: 157 - RNA, 21 nt, Homo sapiens
caagucacuagugguuccguu

SEQ ID NO: 158 - RNA, 22 nt, Homo sapiens
uggcagugucuuagcugguugu

SEQ ID NO: 159 - RNA, 21 nt, Homo sapiens
aucauagaggaaaauccacgu

SEQ ID NO: 160 - RNA, 21 nt, Homo sapiens
aauggcgccacuaggguugug

SEQ ID NO: 161 - RNA, 21 nt, Homo sapiens
aacauagaggaaauuccacgu

SEQ ID NO: 162 - RNA, 23 nt, Homo sapiens
uaaggugcaucuagugcagauag

SEQ ID NO: 163 - RNA, 21 nt, Homo sapiens
aauauaacacagauggccugu

SEQ ID NO: 164 - RNA, 22 nt, Homo sapiens
agagguaguagguugcauaguu

SEQ ID NO: 165 - RNA, 22 nt, Homo sapiens
aaggagcucacagucuauugag

SEQ ID NO: 166 - RNA, 22 nt, Homo sapiens
uguaaacauccuacacucagcu

SEQ ID NO: 167 - RNA, 23 nt, Homo sapiens
uguaaacauccuacacucucagc

SEQ ID NO: 168 - RNA, 21 nt, Mus musculus
ugguagacuauggaacguagg

SEQ ID NO: 169 - RNA, 23 nt, Homo sapiens
cgcauccccuagggcauuggugu

SEQ ID NO: 170 - RNA, 22 nt, Mus musculus
cagugguuuuacccuaugguag

SEQ ID NO: 171 - RNA, 22 nt, Homo sapiens
cacccguagaaccgaccuugcg

SEQ ID NO: 172 - RNA, 22 nt, Homo sapiens
aaucguacagggucauccacuu

SEQ ID NO: 173 - RNA, 23 nt, Homo sapiens
ugugcaaaucuaugcaaaacuga

SEQ ID NO: 174 - RNA, 22 nt, Homo sapiens
cgucaacacuugcugguuuccu

SEQ ID NO: 175 - RNA, 22 nt, Homo sapiens
uuuguucguucggcucgcguga

SEQ ID NO: 176 - RNA, 22 nt, Homo sapiens
aagacgggaggaaagaagggag

SEQ ID NO: 177 - RNA, 21 nt, Homo sapiens
cugaccuaugaauugacagcc

SEQ ID NO: 178 - RNA, 22 nt, Homo sapiens
uggagugugacaaugguguuug

SEQ ID NO: 179 - RNA, 22 nt, Homo sapiens
ugggucuuugcgggcgagauga

SEQ ID NO: 180 - RNA, 22 nt, Homo sapiens
uccauuacacuacccugccucu

SEQ ID NO: 181 - RNA, 22 nt, Homo sapiens
aacuggcccucaaagucccgcu

SEQ ID NO: 182 - RNA, 21 nt, Homo sapiens
caagucacuagugguuccguu

SEQ ID NO: 183 - RNA, 22 nt, Homo sapiens
uggcagugucuuagcugguugu

SEQ ID NO: 184 - RNA, 23 nt, Homo sapiens
caacggaaucccaaaagcagcug

SEQ ID NO: 185 - RNA, 22 nt, Homo sapiens
aucgggaaugucguguccgccc

SEQ ID NO: 186 - RNA, 22 nt, Homo sapiens
aaggagcucacagucuauugag

SEQ ID NO: 187 - RNA, 22 nt, Homo sapiens
uggaauguaaggaagugugugg

SEQ ID NO: 188 - RNA, 21 nt, Homo sapiens
caagucacuagugguuccguu

SEQ ID NO: 189 - RNA, 23 nt, Homo sapiens
agcagcauuguacagggcuauga

SEQ ID NO: 190 - RNA, 21 nt, Homo sapiens
cuagacugaagcuccuugagg

SEQ ID NO: 191 - RNA, 22 nt, Homo sapiens
guucucccaacguaagcccagc

SEQ ID NO: 192 - RNA, 22 nt, Homo sapiens
cguguauuugacaagcugaguu

SEQ ID NO: 193 - RNA, 22 nt, Homo sapiens
uccgucucaguuacuuuauagc

SEQ ID NO: 194 - RNA, 22 nt, Homo sapiens
ugucaguuugucaaauacccca

SEQ ID NO: 195 - RNA, 22 nt, Homo sapiens
agagguaguagguugcauaguu

SEQ ID NO: 196 - RNA, 22 nt, Homo sapiens
aaggagcucacagucuauugag

SEQ ID NO: 197 - RNA, 23 nt, Homo sapiens
cagugcaauaguauugucaaagc

SEQ ID NO: 198 - RNA, 23 nt, Homo sapiens
agcuacauugucugcuggguuuc

SEQ ID NO: 199 - RNA, 22 nt, Homo sapiens
uguaaacauccuacacucagcu

SEQ ID NO: 200 - RNA, 22 nt, Mus musculus
cagugguuuuacccuaugguag

SEQ ID NO: 201 - RNA, 23 nt, Homo sapiens
uguaaacauccuacacucucagc

SEQ ID NO: 202 - RNA, 23 nt, Mus musculus
caaagugcuguucgugcagguag

SEQ ID NO: 203 - RNA, 21 nt, Homo sapiens
uaaagugcugacagugcagau

SEQ ID NO: 204 - RNA, 21 nt, Homo sapiens
gccccugggccuauccuagaa

SEQ ID NO: 205 - RNA, 23 nt, Homo sapiens
aaaagugcuuacagugcagguag

SEQ ID NO: 206 - RNA, 22 nt, Homo sapiens
uguaaacauccucgacuggaag

SEQ ID NO: 207 - RNA, 21 nt, Homo sapiens
ucagugcaugacagaacuugg

SEQ ID NO: 208 - RNA, 22 nt, Homo sapiens
cgucaacacuugcugguuuccu

SEQ ID NO: 209 - RNA, 22 nt, Homo sapiens
uggagugugacaaugguguuug

SEQ ID NO: 210 - RNA, 22 nt, Homo sapiens
ugggucuuugcgggcgagauga

SEQ ID NO: 211 - RNA, 22 nt, Homo sapiens
aagacgggaggaaagaagggag

SEQ ID NO: 212 - RNA, 22 nt, Homo sapiens
uccauuacacuacccugccucu

SEQ ID NO: 213 - RNA, 21 nt, Homo sapiens
cugaccuaugaauugacagcc

SEQ ID NO: 214 - RNA, 22 nt, Homo sapiens
uggcagugucuuagcugguugu

SEQ ID NO: 215 - RNA, 22 nt, Homo sapiens
aacuggcccucaaagucccgcu

SEQ ID NO: 216 - RNA, 21 nt, Homo sapiens
caagucacuagugguuccguu

SEQ ID NO: 217 - RNA, 23 nt, Homo sapiens
ugugcuugcucgucccgcccgca

SEQ ID NO: 218 - RNA, 22 nt, Homo sapiens
uagcagcacguaaauauuggcg

SEQ ID NO: 219 - RNA, 22 nt, Homo sapiens
uccgucucaguuacuuuauagc

SEQ ID NO: 220 - RNA, 22 nt, Homo sapiens
agagguaguagguugcauaguu

SEQ ID NO: 221 - RNA, 23 nt, Homo sapiens
uguaguguuuccuacuuuaugga

SEQ ID NO: 222 - RNA, 22 nt, Homo sapiens
ccucccacacccaaggcuugca

SEQ ID NO: 223 - RNA, 23 nt, Homo sapiens
uguaaacauccuacacucucagc

SEQ ID NO: 224 - RNA, 22 nt, Homo sapiens
uguaaacauccucgacuggaag

SEQ ID NO: 225 - RNA, 22 nt, Homo sapiens
cacgcucaugcacacacccaca

SEQ ID NO: 226 - RNA, 19 nt, Homo sapiens
uggauuuuuggaucaggga

SEQ ID NO: 227 - RNA, 22 nt, Homo sapiens
ugagguaguagguuguaugguu

SEQ ID NO: 228 - RNA, 22 nt, Homo sapiens
uuuguucguucggcucgcguga

SEQ ID NO: 229 - RNA, 21 nt, Homo sapiens
ucagugcaugacagaacuugg

SEQ ID NO: 230 - RNA, 22 nt, Homo sapiens
ugggucuuugcgggcgagauga

SEQ ID NO: 231 - RNA, 22 nt, Homo sapiens
aagacgggaggaaagaagggag

SEQ ID NO: 232 - RNA, 22 nt, Homo sapiens
uggagugugacaaugguguuug

SEQ ID NO: 233 - RNA, 21 nt, Homo sapiens
cugaccuaugaauugacagcc

SEQ ID NO: 234 - RNA, 22 nt, Homo sapiens
uggcagugucuuagcugguugu

SEQ ID NO: 235 - RNA, 22 nt, Homo sapiens
uccauuacacuacccugccucu

SEQ ID NO: 236 - RNA, 21 nt, Homo sapiens
caagucacuagugguuccguu

SEQ ID NO: 237 - RNA, 22 nt, Homo sapiens
aacuggcccucaaagucccgcu